(19) United States
(12) Patent Application Publication
(10) Pub. No.: US 2014/0141436 A1
(43) Pub. Date: May 22, 2014

(54) Methods and Compositions for Very High Resolution Genotyping of HLA

(71) Applicant: Roche Molecular Systems, Inc., Pleasanton, CA (US)

(72) Inventors: Henry Erlich, Oakland, CA (US); Bryan Hoglund, Pleasanton, CA (US); Cherie Holcomb, Oakland, CA (US); Priscilla Moonsamy, Pleasanton, CA (US)

(73) Assignee: Roche Molecular Systems, Inc., Pleasanton, CA (US)

(21) Appl. No.: 13/972,410

(22) Filed: Aug. 21, 2013

Related U.S. Application Data
(63) Continuation-in-part of application No. 12/798,877, filed on Apr. 12, 2010, now abandoned.
Application No. 12/798,877 is a continuation-in-part of application No. 12/245,666, filed on Oct. 3, 2008.
(60) Provisional application No. 61/857,432, filed on Jul. 23, 2013.
(60) Provisional application No. 61/169,465, filed on Apr. 15, 2009.

(51) Int. Cl. C12Q 1/68

(52) U.S. Cl. CPC C12Q 1/6881
USPC 435/6.12; 536/24.33; 536/24.31

(57) ABSTRACT

The invention is a method of determining HLA genotype for HLA-A, HLA-B, HLA-C, DQB1, DRB1, DRB3, DRB4, DRB5, DPA1 and DPB1. Reagents and kits are also disclosed.

### CROSS-REFERENCE TO RELATED APPLICATIONS

This application claims priority to a provisional application Ser. No. 61/857,432 filed on Jul. 23, 2013 and is a Continuation in Part of U.S. patent application Ser. No. 12/798,877 titled “System and method for detection of HLA Variants,” filed on Apr. 12, 2010 which is a Continuation in Part of U.S. patent application Ser. No. 12/245,666, titled “High Resolution, High Throughput HLA Genotyping by Clonal Sequencing”, filed on Oct. 3, 2008; and also claims priority to U.S. Provisional Patent Application Ser. No. 61/169,465, titled “System and Method for Detection of HLA Variants”, filed on Apr. 15, 2009.

### FIELD OF THE INVENTION

The invention provides methods, reagents and systems for detecting and analyzing sequence variants associated with HLA class I and class II loci. The variants may include single nucleotide polymorphisms (SNPs), polymorphic sequence motifs (i.e. complex polymorphisms involving adjacent nucleotides), insertion/deletion variation (referred to as “indels”) and other types of polymorphism or variation known to those of ordinary skill in the related art that can occur in a population of target polynucleotides. The invention also relates to a method of investigating by massively parallel sequencing nucleic acids replicated by polymerase chain reaction (PCR), for the identification of mutations and polymorphisms of both known and unknown sequences. The invention involves using nucleic acid primers specifically designed to amplify a particular region and/or a series of overlapping regions of HLA DNA associated with a particular HLA characteristic or function. Also, the target sites for the primers were selected in part due to a low level of polymorphism enabling consistent amplification of the nucleic acids in a target HLA nucleic acid population which are suspected of containing variants to generate individual amplicons. Thousands of individual HLA amplicons are sequenced in a massively parallel, efficient, and cost effective manner to generate a distribution of the sequence variants found in the populations of amplicons that enables greater sensitivity of detection over previously employed methods.

### BACKGROUND OF THE INVENTION

The Human Leukocyte Antigen (generally referred to as HLA) class I and class II loci are the most polymorphic genes in the human genome, with a complex pattern of patchwork polymorphism (i.e. variants) localized primarily in exon 2 for the class II genes and exons 2 and 3 for the class I genes. For the current HLA typing methods, allele level resolution of HLA alleles, which is clinically important for hematopoietic stem cell transplantation, is technically challenging. Several large scale studies have demonstrated that precise, allele level HLA matching between donor and patient significantly improves overall transplant survival by reducing the incidence and severity of both acute and chronic graft versus host disease and improving the rates of successful engraftment. When, for example, 8 of 8 of the most significant HLA loci are matched vs. 6 of 8, survival after transplant was enhanced by 60% after 12 months.

It is current practice to maintain bone marrow donor registries in which millions of potential donors are HLA typed at low-medium resolution for the A, B, and, in many cases the DRB1 loci. Multiple potentially matched unrelated donors are selected, based on this initial typing, and then typed at the allele level resolution at these and additional loci to identify the donor best matched to the recipient.

Previously, the highest resolution HLA typing of variants has been obtained with fluorescent, Sanger-based DNA sequencing using capillary electrophoresis. However, ambiguities in the HLA typing data can persist due to multiple polymorphisms between alleles and the resultant phase ambiguities when both alleles are amplified and sequenced together. Resolving these ambiguities requires time-consuming approaches such as amplifying and then analyzing the two alleles separately.

Therefore, efficient detection of variation through improved sequencing methods enabled to generate sequence information in parallel from millions of DNA molecules is highly desirable. The clonal sequencing property of this system means that the allelic variants can be sequenced separately, thus allowing the setting of phase of linked polymorphisms in the amplicon. Further, embodiments of improved sequencing methods include target specific high throughput sequencing techniques which have read lengths of about 250 nucleotides, about 400 nucleotides, or >400 nucleotides that enable complete sequence coverage of important HLA regions. For example, the target specific high throughput sequencing technologies employing HLA specific primers of the presently described invention are capable of setting the phase of the linked polymorphisms within an exon and make possible the unambiguous determination of the sequence of each HLA allele.

The unique property of HLA makes it useful in a multitude of applications. HLA genotyping is the basis of histocompatibility testing for stem cell and solid organ transplantation. Other applications include forensics (reviewed in Wu, Y. Y. and Csako, G., (2006) Rapid and/or high-throughput genotyping for human red blood cell, platelet and leukocyte antigens, and forensic applications, Clin. Chico. Acta. 363:165); and more recently, prenatal diagnosis (U.S. Provisional App. Ser. No. 61/821,620). Furthermore, particular HLA alleles have been shown to have statistical association with disease. Therefore disease association studies also require determination of HLA alleles in the patient population.

The HLA complex spans approximately 3.5 million base pairs on the short arm of chromosome 6. The major regions are the Class I and Class II regions. The Class I genes are HLA-A, HLA-B, and HLA-C and the major Class II genes are HLA-DP, HLA-DQ and HLA-DR. Polymorphisms that are expressed at the protein level are reflected in the amino acid sequence of the HLA antigen and therefore are of great interest for tissue typing for transplantation. These polymorphisms are localized primarily in exon 2 for the Class II genes and exons 2 and 3 for the Class I genes. However, for other purposes, e.g., forensics or certain aspects of prenatal diagnosis, all polymorphisms, including the silent changes (nucleotide changes not resulting in an amino acid change, as well as changes in introns and other non-coding regions of the HLA genes) are useful.

HLA typing has become increasingly precise as new HLA alleles are being discovered. In every application of HLA typing, the most precise determination of the HLA genotype (down to the level of individual alleles at as many loci as possible) offers the best chance of a good outcome, e.g., successful transplantation, correct identification of a person or accurate prenatal diagnosis. It is therefore desirable to develop tools for precise genotyping of the HLA locus.

### SUMMARY OF THE INVENTION

In one embodiment, the invention is a method of determining HLA genotype of at least one individual for HLA-A, HLA-B, HLA-C, DQB1, DRB1, DRB3, DRB4, DRB5, DPA1 and DPB1 the method comprising amplifying sequences of HLA-A, Exons 1, 2, 3, 4, 5, HLA-B, Exons 1, 2, 3, 4, 5, HLA-C, Exons 1, 2, 3, 4, 5, 6, 7, DQB1, Exons 2, 3, DRB1, Exons 2, 3, DRB3, Exons 2, 3, DRB4, Exons 2, 3, DRB5, Exons 2, 3, DPA1, Exon 2, and DPB1, Exon 2 using oligonucleotides comprising the HLA hybridizing sequences of SEQ ID NOs: 23-144 to generate HLA amplicons; determining the sequence of the HLA amplicons; and assigning the HLA alleles to the individual thereby determining the HLA genotype of the individual.

In another embodiment, the invention is a kit comprising primer pairs for obtaining HLA amplicons for determining HLA genotype of at least one individual for HLA-A, HLA-B, HLA-C, DRB1, DRB3, DRB4, DRB5 and DPA1 comprising or consisting of fusion oligonucleotides having the HLA hybridizing region of SEQ ID NOs: 129-144.

In yet another embodiment, the invention is a kit comprising primer pairs for obtaining HLA amplicons for determining HLA genotype of at least one individual for HLA-A, HLA-B, HLA-C, DQB1, DRB1, DRB3, DRB4, DRB5, DPA1 and DPB1 comprising or consisting of fusion oligonucleotides having the HLA hybridizing region of SEQ ID NOs: 23-144.

In yet another embodiment, the invention is a method of obtaining HLA amplicons for determining HLA genotype of at least one individual at HLA-A, Exons 1, 2, 3, 4, 5, HLA-B, Exons 1, 2, 3, 4, 5, HLA-C, Exons 1, 2, 3, 4, 5, 6, 7, DQB1, Exons 2, 3, DRB1, Exons 2, 3, DRB3, Exons 2, 3, DRB4, Exons 2, 3, DRB5, Exons 2, 3, DPA1, Exon 2, and DPB1, Exon 2 comprising the steps of: amplifying a plurality of first amplicons from a sample derived from the individual, wherein the first amplicons are amplified with a plurality of pairs of nucleic acid primers selected from SEQ ID NOs: 23-144; amplifying the first amplicons to produce a plurality of populations of second amplicons, wherein each population of second amplicons is clonally amplified from one of the first amplicons; sequencing the plurality of populations of second amplicons to generate a nucleic acid sequence composition for each of the plurality of second amplicons; and assigning the HLA alleles to the individual thereby determining the HLA genotype of the individual. The said plurality of pairs of nucleic acid primers may comprise SEQ ID NOs: 23-68 and 129-144 or SEQ ID NOs: 69-144.

In yet another embodiment, the invention is a set of oligonucleotides for obtaining HLA amplicons at HLA-A, HLA-B, HLA-C, DRB1, DRB3, DRB4, DRB5 and DPA1 comprising one or more pairs of fusion oligonucleotides having the HLA hybridizing region of SEQ ID NO: 129-144. In variations of this embodiment, the invention is a set comprising or consisting of oligonucleotides having the HLA hybridizing region of SEQ ID NO: 129-144; 23-144, 23-68 and 129-144 or 69-144.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 160 appears first in FIG. 1). All of these conventions, however, are intended to be typical or illustrative, rather than limiting.

FIG. 1 is a functional block diagram of one embodiment of a sequencing instrument under computer control and a reaction substrate.

FIG. 2 is a simplified graphical representation of the relationship between the first amplicons to the HLA-A, B, and C genomic regions (exon and intron structure).

FIG. 3 is a simplified graphical representation of the relationship between the first amplicons to the DPA1, DPB1, and DQA1 HLA regions.

FIG. 4 is a simplified graphical representation of the relationship between the first amplicons to the DQB1, and DRB1 HLA regions.

FIG. 5 shows results of very high resolution (VHR) typing of HLA loci using the methods and compositions of the invention.

### DETAILED DESCRIPTION OF THE INVENTION

### Definitions

The term “flowgram” generally refers to a graphical representation of sequence data generated by SBS methods, particularly pyrophosphate based sequencing methods (also referred to as “pyrosequencing”) and may be referred to more specifically as a “pyrogram”.

The term “read” or “sequence read” as used herein generally refers to the entire sequence data obtained from, a single nucleic acid template molecule or a population of a plurality of substantially identical copies of the template nucleic acid molecule.

The terms “run” or “sequencing run” as used herein generally refer to a series of sequencing reactions performed in a sequencing operation of one or more template nucleic acid molecules.

The term “flow” as used herein generally refers to a serial or iterative cycle of addition of solution to an environment comprising a template nucleic acid molecule, where the solution may include a nucleotide species for addition to a nascent molecule or other reagent, such as buffers or enzymes that may be employed in a sequencing reaction or to reduce carryover or noise effects from previous flow cycles of nucleotide species.

The term “flow cycle” as used herein generally refers to a sequential series of flows where a nucleotide species is flowed once during the cycle (i.e. a flow cycle may include a sequential addition in the order of T, A, C, G nucleotide species, although other sequence combinations are also considered part of the definition). Typically, the flow cycle is a repeating cycle having the same sequence of flows from cycle to cycle.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

The term “test fragment” or “TF” as used herein generally refers to a nucleic acid element of known sequence composition that may be employed for quality control, calibration, or other related purposes.

A “nascent molecule” generally refers to a DNA strand which is being extended by the template-dependent DNA polymerase by incorporation of nucleotide species which are complementary to the corresponding nucleotide species in the template molecule.

The terms “template nucleic acid”, “template molecule”, “target nucleic acid”, or “target molecule” generally refer to a nucleic acid molecule that is the subject of a sequencing reaction from which sequence data or information is generated.

The term “nucleotide species” as used herein generally refers to the identity of a nucleic acid monomer including purines (Adenine, Guanine) and pyrimidines (Cytosine, Uracil, Thymine) typically incorporated into a nascent nucleic acid molecule.

The term “monomer repeat” or “homopolymers” as used herein generally refers to two or more sequence positions comprising the same nucleotide species (i.e. a repeated nucleotide species).

The term “homogeneous extension” as used herein, generally refers to the relationship or phase of an extension reaction where each member of a population of substantially identical template molecules is homogenously performing the same extension step in the reaction.

The term “completion efficiency” as used herein generally refers to the percentage of nascent molecules that are properly extended during a given flow.

The term “incomplete extension rate” as used herein generally refers to the ratio of the number of nascent molecules that fail to be properly extended over the number of all nascent molecules.

The term “genomic library” or “shotgun library” as used herein generally refers to a collection of molecules derived from and/or representing an entire genome (i.e. all regions of a genome) of an organism or individual.

The term “amplicon” as used herein generally refers to selected amplification products, such as those produced from Polymerase Chain Reaction or Ligase Chain Reaction techniques.

The term “variant” or “allele” as used herein generally refers to one of a plurality of species each encoding a similar sequence composition, but with a degree of distinction from each other. The distinction may include any type of genetic variation known to those of ordinary skill in the related art, that include, but are not limited to, polymorphisms such as single nucleotide polymorphisms (SNPs), insertions or deletions (the combination of insertion/deletion events are also referred to as “indels”), differences in the number of repeated sequences (also referred to as tandem repeats), and structural variations. For HLA alleles, typically, multiple genetic differences constitute an allele (i.e., most alleles differ from one another by more than one base). In reference to HLA genes, an allele is defined by the use of all of the digits in a current allele name. Examples include A*01:01:01:01 and A*02:07 (designations based on the IMGT/HLA database), see Nunes, E. et al., (2011) Definitions of histocompatibility typing terms, Blood, 118:e180.

The term “allele frequency” or “allelic frequency” as used herein generally refers to the proportion of all variants in a population that is comprised of a particular variant.

The term “key sequence” or “key element” as used herein generally refers to a nucleic acid sequence element (typically of about 4 sequence positions, i.e., TGAC or other combination of nucleotide species) associated with a template nucleic acid molecule in a known location (i.e., typically included in a ligated adaptor element) comprising known sequence composition that is employed as a quality control reference for sequence data generated from template molecules. The sequence data passes the quality control if it includes the known sequence composition associated with a Key element in the correct location.

The term “keypass” or “keypass well” as used herein generally refers to the sequencing of a full length nucleic acid test sequence of known sequence composition (i.e., a “test fragment” or “TF” as referred to above) in a reaction well, where the accuracy of the sequence derived from keypass test sequence is compared to the known sequence composition and used to measure of the accuracy of the sequencing and for quality control. In typical embodiments, a proportion of the total number of wells in a sequencing run will be keypass wells which may, in some embodiments, be regionally distributed.

The term “blunt end” as used herein is interpreted consistently with the understanding of one of ordinary skill in the related art, and generally refers to a linear double stranded nucleic acid molecule having an end that terminates with a pair of complementary nucleotide base species, where a pair of blunt ends is typically compatible for ligation to each other.

The term “sticky end” or “overhang” as used herein is interpreted consistently with the understanding of one of ordinary skill in the related art, and generally refers to a linear double stranded nucleic acid molecule having one or more unpaired nucleotide species at the end of one strand of the molecule, where the unpaired nucleotide species may exist on either strand and include a single base position or a plurality of base positions (also sometimes referred to as “cohesive end”).

The term “bead” or “bead substrate” as used herein generally refers to any type of bead of any convenient size and fabricated from any number of known materials such as cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (as described, e.g., in Merrifield, Biochemistry 1964, 3, 1385-1390), polyacrylamides, latex gels, polystyrene, dextran, rubber, silicon, plastics, nitrocellulose, natural sponges, silica gels, control pore glass, metals, cross-linked dextrans (e.g., Sephadex®) agarose gel (Sepharose), and other solid phase bead supports known to those of skill in the art.

The term “clonal” in the context of “clonal analysis” refers to separately analyzing an aggregate or population of molecules all derived from a single molecule. For example, “clonal sequencing” refers to individually sequencing each amplicon that was derived from the same target region.

The term “deep sequencing” refers to a sequencing method wherein the target sequence is read multiple times in the single test. A single deep sequencing run is composed of a multitude of sequencing reactions run on the same target sequence and each, generating independent sequence readout.

The term “polymorphism” refers to the condition in which two or more variants of a genomic sequence, or the encoded amino acid sequence, can be found in a population. A “single nucleotide polymorphism,” (SNP) is a polymorphism where the variation in the sequence consists of a single polymorphic nucleotide position in the genomic sequence.

The term “genotype” refers to a combination of one or more alleles of one or more genes contained in an individual or a sample derived from the individual.

The term “haplotype” refers to a combination of one or more alleles of one or more genes present on the same chromosome of an individual.

The terms “determining HLA genotype,” “HLA genotyping” and “performing HLA genotyping” are used interchangeably to refer to determining a selected combination of HLA alleles in a subject. For example, determining HLA genotype refers to identifying one or more of the polymorphic residues (allelic determinants) present in one or more exons, e.g., exons 2, 3 and 4 of one or more of the HLA genes: HLA-A, HLA-B, HLA-C, DRB1, DRB3, DRB4, DRB5, DPB1, DPA1, DQA1 and DQB1.

The term “low resolution HLA typing” refers to typing at the serological or protein level.

The terms “high” and “very high resolution typing” refer to typing at the nucleic acid level that is capable of distinguishing among HLA proteins indistinguishable at the serological level yet encoded by different nucleotide sequences. As used herein, the distinction between high and very high resolution is in the number of HLA loci probed by the test.

The term “target region” refers to a region of a nucleic acid sequence that is to be analyzed.

The term “nucleic acid” refers to polymers of nucleotides (e.g., ribonucleotides or deoxyribonucleotides) both natural and non-natural. The term is not limited by length (e.g., number of monomers) of the polymer. A nucleic acid may be single-stranded or double-stranded and will generally contain 5′-3′ phosphodiester bonds, although in some cases, nucleotide analogs may have other linkages. Nucleic acids may include naturally occurring bases (adenosine, guanidine, cytosine, uracil and thymidine) as well as non-natural bases. The term “non-natural nucleotide” or “modified nucleotide” refers to a nucleotide that contains a modified nitrogenous base, sugar or phosphate group, or that incorporates a non-natural moiety in its structure. Examples of non-natural nucleotides include dideoxynucleotides, biotinylated, aminated, deaminated, alkylated, benzylated and fluorophor-labeled nucleotides.

The term “primer” refers to a short nucleic acid (an oligonucleotide) that acts as a point of initiation of DNA synthesis by a nucleic acid polymerase under suitable conditions that typically include an appropriate buffer, the presence of nucleic acid precursors and one or more optional cofactors and a suitable temperature. A primer typically includes at least one target-hybridized region that is at least substantially complementary to the target sequence. This region is typically of about 15 to about 40 nucleotides in length.

The term “adapter region” or “adapter” of a primer refers to the region of a primer typically located to the 5′ of the target-hybridizing region. Typically the adapter serves a function in a subsequent analysis step. For example, the adapter may hybridize to an oligonucleotide conjugated to a microparticle or other solid surface used for amplification, e.g., emulsion PCR. The adapter can also serve as a binding site for a primer used in subsequent steps, e.g., a sequencing primer. The adapter region is typically from 15 to 30 nucleotides in length.

The terms “individual identifier tag,” “identification tag,” “multiplex identification tag” or “MID” are used interchangeably herein to refer to a region of a primer that serves as a marker of the DNA obtained from a particular sample.

The term “sample” refers to any composition containing or presumed to contain nucleic acid from an individual. In the context of the present invention, any type of body sample may be used, including without limitation, skin, buccal swab, tissue biopsy, plasma, serum, whole blood and blood components, saliva, urine, tears, seminal fluid, vaginal fluids and other fluids and tissues, including paraffin embedded tissues or tissues collected and preserved in the course of a forensic investigation. Samples also may include constituents and components of in vitro cultures of cells obtained from an individual. In some specific applications, e.g., HLA typing in prenatal diagnosis, the sample may be maternal blood and fractions derived therefrom, e.g., blood plasma; amniotic fluid or a fetal tissue or fetal fluid.

The term “four primer amplification” refers to a polymerase chain reaction amplification in which one set of forward and reverse primers (sometimes referred to as “inner primers”) contains sequences on the 3′ ends that are specific to the genomic target and sequences on their 5′ ends that are identical to the sequences on the 3′ ends of a second set of forward and reverse primers (sometimes referred to as “outer primers”). The inner and outer primers can be present either in the same PCR or in sequential PCRs (i.e., a PCR with outer primers performed after that with inner primers). The sequences on the 3′ ends of the forward and reverse outer primers (and thus the 5′ ends of the forward and reverse inner primers) are different from each other.

The invention provides methods and composition for Very High Resolution (VHR) genotyping of the HLA class I and class II loci. In one embodiment, the invention is a set of oligonucleotides that specifically hybridize to certain regions throughout the HLA locus to achieve VHR genotyping of the locus. In other embodiments, the invention is a method of VHR genotyping of the HLA locus using the oligonucleotides disclosed herein. In yet other embodiments, the invention is an article of manufacture (e.g., a kit) comprising the oligonucleotides disclosed herein.

Some exemplary embodiments of systems and methods associated with sample preparation and processing, generation of sequence data, and analysis of sequence data are generally described below, some or all of which are amenable for use with embodiments of the presently described invention. In particular, the exemplary embodiments of systems and methods for preparation of template nucleic acid molecules, amplification of template molecules, generating target specific amplicons and/or genomic libraries, sequencing methods and instrumentation, and computer systems are described.

In typical embodiments, the nucleic acid molecules derived from an experimental or diagnostic sample must be prepared and processed from its raw form into template molecules amenable for high throughput sequencing. The processing methods may vary from application to application, resulting in template molecules comprising various characteristics. For example, in some embodiments of high throughput sequencing, it is preferable to generate template molecules with a sequence or read length that is at least the length a particular sequencing method can accurately produce sequence data for. In the present example, the length may include a range of about 25-30 base pairs, about 50-100 base pairs, about 200-300 base pairs, about 350-500 base pairs, greater than 500 base pairs, or other length amenable for a particular sequencing application. In some embodiments, nucleic acids from a sample, such as a genomic sample, are fragmented using a number of methods known to those of ordinary skill in the art. In preferred embodiments, methods that randomly fragment (i.e. do not select for specific sequences or regions) nucleic acids and may include what is referred to as nebulization or sonication methods. It will, however, be appreciated that other methods of fragmentation, such as digestion using restriction endonucleases, may be employed for fragmentation purposes. Also in the present example, some processing methods may employ size selection methods known in the art to selectively isolate nucleic acid fragments of the desired length.

Also, it is preferable in some embodiments to associate additional functional elements with each template nucleic acid molecule. The elements may be employed for a variety of functions including, but not limited to, primer sequences for amplification and/or sequencing methods, quality control elements (i.e. such as Key elements or other type of quality control element), unique identifiers (also referred to as a multiplex identifier or “MID”) that encode various associations such as with a sample of origin or patient, or other functional element.

For example, some embodiments of the described invention comprise associating one or more embodiments of an MID element having a known and identifiable sequence composition with a sample, and coupling the embodiments of MID element with template nucleic acid molecules from the associated samples. The MID coupled template nucleic acid molecules from a number of different samples are pooled into a single “Multiplexed” sample or composition that can then be efficiently processed to produce sequence data for each MID coupled template nucleic acid molecule. The sequence data for each template nucleic acid is de-convoluted to identify the sequence composition of coupled MID elements and association with sample of origin identified. In the present example, a multiplexed composition may include representatives from about 384 samples, about 96 samples, about 50 samples, about 20 samples, about 16 samples, about 10 samples, or other number of samples. Each sample may be associated with a different experimental condition, treatment, species, or individual in a research context. Similarly, each sample may be associated with a different tissue, cell, individual, condition, or treatment in a diagnostic context. Those of ordinary skill in the related art will appreciate that the numbers of samples listed above are for the purposes of example and thus should not be considered limiting.

In preferred embodiments, the sequence composition of each MID element is easily identifiable and resistant to introduced error from sequencing processes. Some embodiments of MID element comprise a unique sequence composition of nucleic acid species that has minimal sequence similarity to a naturally occurring sequence. Alternatively, embodiments of a MID element may include some degree of sequence similarity to naturally occurring sequence.

Also, in preferred embodiments the position of each MID element is known relative to some feature of the template nucleic acid molecule and/or adaptor elements coupled to the template molecule. Having a known position of each MID is useful for finding the MID element in sequence data and interpretation of the MID sequence composition for possible errors and subsequent association with the sample of origin. For example, some features useful as anchors for positional relationship to MID elements may include, but are not limited to, the length of the template molecule (i.e. the MID element is known to be so many sequence positions from the 5′ or 3′ end), recognizable sequence markers such as a Key element and/or one or more primer elements positioned adjacent to a MID element. In the present example, the Key and primer elements generally comprise a known sequence composition that typically does not vary from sample to sample in the multiplex composition and may be employed as positional references for searching for the MID element. An analysis algorithm implemented by application 135 may be executed on computer 130 to analyze generated sequence data for each MID coupled template to identify the more easily recognizable Key and/or primer elements, and extrapolate from those positions to identify a sequence region presumed to include the sequence of the MID element. Application 135 may then process the sequence composition of the presumed region and possibly some distance away in the flanking regions to positively identify the MID element and its sequence composition.

Some or all of the described functional elements may be combined into adaptor elements that are coupled to nucleotide sequences in certain processing steps. For example, some embodiments may associate priming sequence elements or regions comprising complementary sequence composition to primer sequences employed for amplification and/or sequencing. Further, the same elements may be employed for what may be referred to as “strand selection” and immobilization of nucleic acid molecules to a solid phase substrate. In some embodiments, two sets of priming sequence regions (hereafter referred to as priming sequence A, and priming sequence B) may be employed for strand selection, where only single strands having one copy of priming sequence A and one copy of priming sequence B is selected and included as the prepared sample. In alternative embodiments, design characteristics of the adaptor elements eliminate the need for strand selection. The same priming sequence regions may be employed in methods for amplification and immobilization where, for instance, priming sequence B may be immobilized upon a solid substrate and amplified products are extended therefrom.

Additional examples of sample processing for fragmentation, strand selection, and addition of functional elements and adaptors are described in U.S. patent application Ser. No. 10/767,894, titled “Method for preparing single-stranded DNA libraries”, filed Jan. 28, 2004; U.S. patent application Ser. No. 12/156,242, titled “System and Method for Identification of Individual Samples from a Multiplex Mixture”, filed May 29, 2008; and U.S. patent application Ser. No. 12/380,139, titled “System and Method for Improved Processing of Nucleic Acids for Production of Sequencable Libraries”, filed Feb. 23, 2009, each of which is hereby incorporated by reference herein in its entirety for all purposes.

Various examples of systems and methods for performing amplification of template nucleic acid molecules to generate populations of substantially identical copies are described. It will be apparent to those of ordinary skill that it is desirable in some embodiments of SBS to generate many copies of each nucleic acid element to generate a stronger signal when one or more nucleotide species is incorporated into each nascent molecule associated with a copy of the template molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above) and Polymerase Chain Reaction (PCR) methods, each of the techniques are applicable for use with the presently described invention. One PCR technique that is particularly amenable to high throughput applications include what are referred to as emulsion PCR methods (also referred to as emPCR™ methods).

Typical embodiments of emulsion PCR methods include creating a stable emulsion of two immiscible substances creating aqueous droplets within which reactions may occur. In particular, the aqueous droplets of an emulsion amenable for use in PCR methods may include a first fluid, such as a water based fluid suspended or dispersed as droplets (also referred to as a discontinuous phase) within another fluid, such as a hydrophobic fluid (also referred to as a continuous phase) that typically includes some type of oil. Examples of oil that may be employed include, but are not limited to, mineral oils, silicone based oils, or fluorinated oils.

Further, some emulsion embodiments may employ surfactants that act to stabilize the emulsion, which may be particularly useful for specific processing methods such as PCR. Some embodiments of surfactant may include one or more of a silicone or fluorinated surfactant. For example, one or more non-ionic surfactants may be employed that include, but are not limited to, sorbitan monooleate (also referred to as Span® 80), polyoxyethylenesorbitsan monooleate (also referred to as Tween® 80), or in some preferred embodiments, dimethicone copolyol (also referred to as Abil® EM90), polysiloxane, polyalkyl polyether copolymer, polyglycerol esters, poloxamers, and PVP/hexadecane copolymers (also referred to as Unimer U-151), or in more preferred embodiments, a high molecular weight silicone polyether in cyclopentasiloxane (also referred to as DC 5225C available from Dow Corning).

The droplets of an emulsion may also be referred to as compartments, microcapsules, microreactors, microenvironments, or other name commonly used in the related art. The aqueous droplets may range in size depending on the composition of the emulsion components or composition, contents contained therein, and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a solid substrate such as a bead for attachment of template and amplified copies of the template, amplified copies complementary to the template, or combination thereof. Further, the solid substrate may be enabled for attachment of other type of nucleic acids, reagents, labels, or other molecules of interest.

Embodiments of an emulsion useful with the presently described invention may include a very high density of droplets or microcapsules enabling the described chemical reactions to be performed in a massively parallel way. Additional examples of emulsions employed for amplification and their uses for sequencing applications are described in U.S. Pat. Nos. 7,638,276; 7,622,280 and 7,842,457; and U.S. patent application Ser. No. 10/767,899; each of which is hereby incorporated by reference herein in its entirety for all purposes.

Also embodiments sometimes referred to as Ultra-Deep Sequencing, generate target specific amplicons for sequencing may be employed with the presently described invention that include using sets of specific nucleic acid primers to amplify a selected target region or regions from a sample comprising the target nucleic acid. Further, the sample may include a population of nucleic acid molecules that are known or suspected to contain sequence variants comprising sequence composition associated with a research or diagnostic utility where the primers may be employed to amplify and provide insight into the distribution of sequence variants in the sample. For example, a method for identifying a sequence variant by specific amplification and sequencing of multiple alleles in a nucleic acid sample may be performed. The nucleic acid is first subjected to amplification by a pair of PCR primers designed to amplify a region surrounding the region of interest or segment common to the nucleic acid population. Each of the products of the PCR reaction (first amplicons) is subsequently further amplified individually in separate reaction vessels such as an emulsion based vessel described above. The resulting amplicons (referred to herein as second amplicons), each derived from one member of the first population of amplicons, are sequenced and the collection of sequences are used to determine an allelic frequency of one or more variants present. Importantly, the method does not require previous knowledge of the variants present and can typically identify variants present at <1% frequency in the population of nucleic acid molecules.

Some advantages of the described target specific amplification and sequencing methods include a higher level of sensitivity than previously achieved. Further, embodiments that employ high throughput sequencing instrumentation, such as for instance embodiments that employ what is referred to as a PicoTiterPlate® array (also sometimes referred to as a PTP® plate or array) of wells provided by 454 Life Sciences Corporation, the described methods can be employed to generate sequence composition for over 100,000, over 300,000, over 500,000, or over 1,000,000 nucleic acid regions per run or experiment and may depend, at least in part, on user preferences such as lane configurations enabled by the use of gaskets, etc. Also, the described methods provide a sensitivity of detection of low abundance alleles which may represent 1% or less of the allelic variants. Another advantage of the methods includes generating data comprising the sequence of the analyzed region. Importantly, it is not necessary to have prior knowledge of the sequence of the locus being analyzed.

Additional examples of target specific amplicons for sequencing are described in U.S. patent application Ser. No. 11/104,781, titled “Methods for determining sequence variants using ultra-deep sequencing”, filed Apr. 12, 2005; PCT Patent Application Serial No. US 2008/003424, titled “System and Method for Detection of HIV Drug Resistant Variants”, filed Mar. 14, 2008; and U.S. Pat. No. 7,888,034, each of which is hereby incorporated by reference herein in its entirety for all purposes.

Further, embodiments of sequencing may include Sanger type techniques, techniques generally referred to as Sequencing by Hybridization (SBH), Sequencing by Ligation (SBL), or Sequencing by Incorporation (SBI) techniques. Further, the sequencing techniques may include what is referred to as colony sequencing techniques; nanopore, waveguide and other single molecule detection techniques; or reversible terminator techniques. As described above, a preferred technique may include Sequencing by Synthesis methods. For example, some SBS embodiments sequence populations of substantially identical copies of a nucleic acid template and typically employ one or more oligonucleotide primers designed to anneal to a predetermined, complementary position of the sample template molecule or one or more adaptors attached to the template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species. Alternatively, in some embodiments the primer/template complex is presented with a plurality of nucleotide species of interest (typically A, G, C, and T) at once, and the nucleotide species that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. It will also be appreciated that the process of adding a nucleotide species to the end of a nascent molecule is substantially the same as that described above for addition to the end of a primer.

As described above, incorporation of the nucleotide species can be detected by a variety of methods known in the art, e.g. by detecting the release of pyrophosphate (PPi) (examples described in U.S. Pat. Nos. 6,210,891; 6,258,568; and 6,828,100, each of which is hereby incorporated by reference herein in its entirety for all purposes), or via detectable labels bound to the nucleotides. Some examples of detectable labels include but are not limited to mass tags and fluorescent or chemiluminescent labels. In typical embodiments, unincorporated nucleotides are removed, for example by washing. Further, in some embodiments the unincorporated nucleotides may be subjected to enzymatic degradation such as, for instance, degradation using the apyrase or pyrophosphatase enzymes as described in U.S. patent application Ser. Nos. 12/215,455, titled “System and Method for Adaptive Reagent Control in Nucleic Acid Sequencing”, filed Jun. 27, 2008; and 12/322,284, titled “System and Method for Improved Signal Detection in Nucleic Acid Sequencing”, filed Jan. 29, 2009; each of which is hereby incorporated by reference herein in its entirety for all purposes.

In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Repeated cycles of nucleotide addition, extension, signal acquisition, and washing result in a determination of the nucleotide sequence of the template strand. Continuing with the present example, a large number or population of substantially identical template molecules (e.g. 10³, 10⁴, 10⁵, 10⁶ or 10⁷ molecules) are typically analyzed simultaneously in any one sequencing reaction, in order to achieve a signal which is strong enough for reliable detection.

In addition, it may be advantageous in some embodiments to improve the read length capabilities and qualities of a sequencing process by employing what may be referred to as a “paired-end” sequencing strategy. For example, some embodiments of sequencing method have limitations on the total length of molecule from which a high quality and reliable read may be generated. In other words, the total number of sequence positions for a reliable read length may not exceed 25, 50, 100, or 500 bases depending on the sequencing embodiment employed. A paired-end sequencing strategy extends reliable read length by separately sequencing each end of a molecule (sometimes referred to as a “tag” end) that comprise a fragment of an original template nucleic acid molecule at each end joined in the center by a linker sequence. The original positional relationship of the template fragments is known and thus the data from the sequence reads may be re-combined into a single read having a longer high quality read length. Further examples of paired-end sequencing embodiments are described in U.S. Pat. No. 7,601,499, titled “Paired end sequencing”; and in U.S. patent application Ser. No. 12/322,119, titled “Paired end sequencing”, filed Jan. 28, 2009, each of which is hereby incorporated by reference herein in its entirety for all purposes.

Some examples of SBS apparatus may implement some or all of the methods described above and may include one or more of a detection device such as a charge coupled device (i.e., CCD camera) or a confocal type architecture, a microfluidics chamber or flow cell, a reaction substrate, and/or a pump and flow valves. Taking the example of pyrophosphate based sequencing, embodiments of an apparatus may employ a chemiluminescent detection strategy that produces an inherently low level of background noise.

In some embodiments, the reaction substrate for sequencing may include what is referred to as a PTP® array available from 454 Life Sciences Corporation, as described above, formed from a fiber optics faceplate that is acid-etched to yield hundreds of thousands or more of very small wells each enabled to hold a population of substantially identical template molecules (i.e., some preferred embodiments comprise about 3.3 million wells on a 70×75 mm PTP® array at a 35 urn well to well pitch). In some embodiments, each population of substantially identical template molecule may be disposed upon a solid substrate, such as a bead, each of which may be disposed in one of said wells. For example, an apparatus may include a reagent delivery element for providing fluid reagents to the PTP plate holders, as well as a CCD type detection device enabled to collect photons of light emitted from each well on the PTP plate. An example of reaction substrates comprising characteristics for improved signal recognition is described in U.S. Pat. No. 7,682,816, which is hereby incorporated by reference herein in its entirety for all purposes. Further examples of apparatus and methods for performing SBS type sequencing and pyrophosphate sequencing are described in U.S. Pat. Nos. 7,323,305 and 7,682,816, both of which are incorporated by reference above.

In addition, systems and methods may be employed that automate one or more sample preparation processes, such as the emPCR® process described above. For example, automated systems may be employed to provide an efficient solution for generating an emulsion for emPCR processing, performing PCR Thermocycling operations, and enriching for successfully prepared populations of nucleic acid molecules for sequencing. Examples of automated sample preparation systems are described in U.S. Pat. No. 7,927,797, which is hereby incorporated by reference herein in its entirety for all purposes.

Also, the systems and methods of the presently described embodiments of the invention may include implementation of some design, analysis, or other operation using a computer readable medium stored for execution on a computer system. For example, several embodiments are described in detail below to process detected signals and/or analyze data generated using SBS systems and methods where the processing and analysis embodiments are implementable on computer systems.

An exemplary embodiment of a computer system for use with the presently described invention may include any type of computer platform such as a workstation, a personal computer, a server, or any other present or future computer. It will, however, be appreciated by one of ordinary skill in the art that the aforementioned computer platforms as described herein are specifically configured to perform the specialized operations of the described invention and are not considered general purpose computers. Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provides one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof.

A processor may include a commercially available processor such as a Celeron®, Core®, or Pentium® processor made by Intel Corporation, a SPARC® processor made by Sun Microsystems, an Athlon®, Sempron®, Phenom®, or Opteron® processor made by AMD corporation, or it may be one of other processors that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows®-type operating system (such as Windows® XP, Windows Vista®, or Windows®-7) from the Microsoft Corporation; the Mac OS X operating system from Apple Computer Corp. (such as Mac OS X v10.6 “Snow Leopard” operating systems); a Unix® or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium, such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also, a computer may include one or more library files, experiment data files, and an internet client stored in system memory. For example, experiment data could include data related to one or more experiments or assays such as detected signal values, or other values associated with one or more SBS experiments or processes. Additionally, an internet client may include an application enabled to accesses a remote service on another computer using a network and may for instance comprise what are generally referred to as “Web Browsers”. In the present example, some commonly employed web browsers include Microsoft® Internet Explorer 8 available from Microsoft Corporation, Mozilla Firefox®6 from the Mozilla Corporation, Safari 4 from Apple Computer Corp., Google Chrome from the Google® Corporation, or other type of web browser currently known in the art or to be developed in the future. Also, in the same or other embodiments an internet client may include, or could be an element of, specialized software applications enabled to access remote information via a network such as a data processing application for biological applications.

A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control infomiation traffic to and from hardware and/or software systems. For example, firewalls may comprise hardware or software elements or some combination thereof and are typically designed to enforce security policies put in place by users, such as for instance network administrators, etc.

### b. Embodiments of the Presently Described Invention

As described above, embodiments of the invention relate to methods of identifying or diagnosing a number of sequence variants associated with HLA (e.g., allelic variants, single nucleotide polymorphism variants, indel variants) by the identification of specific DNA. Examples of HLA alleles are described in Mason and Parham (1998) Tissue Antigens 51: 417-66, which lists HLA-A, HLA-B, and HLA-C alleles and Marsh et al. (1992) Hum. Immunol. 35:1, which list HLA class II alleles for DRA, DRB, DQA 1, DQB1, DPA1, and DPB1.

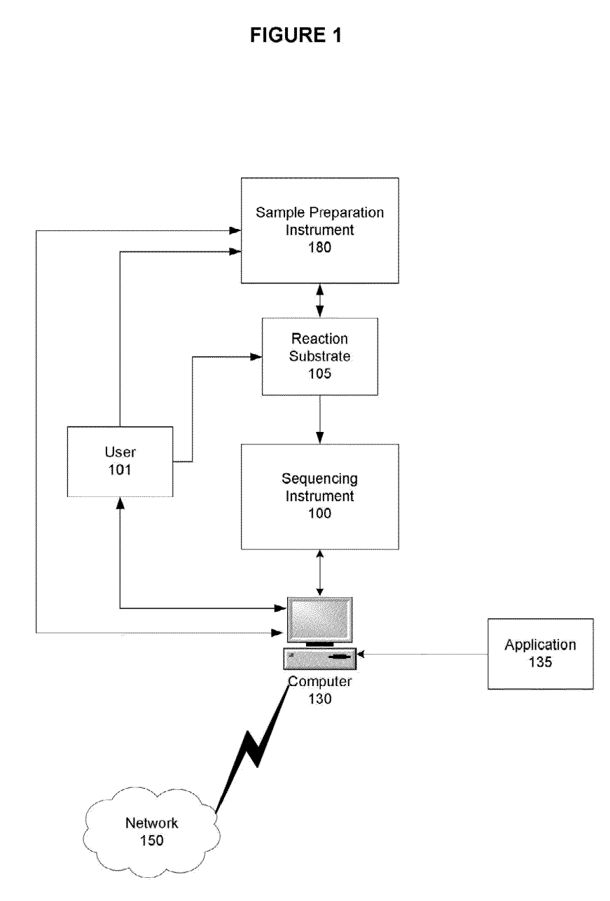

Typically, one or more instrument elements may be employed that automate one or more process steps. For example, embodiments of a sequencing method may be executed using instrumentation to automate and carry out some or all process steps. FIG. 1 provides an illustrative example of sequencing instrument 100 that for sequencing processes requiring capture of optical signals typically comprise an optic subsystem and a fluidic subsystem for execution of sequencing reactions and data capture that occur on reaction substrate 105. It will, however, be appreciated that for sequencing processes requiring other modes of data capture (i.e. PH, temperature, electrochemical, etc.) a subsystem for the mode of data capture may be employed which are known to those of ordinary skill in the related art.

Embodiments of sequencing instrument 100 employed to execute sequencing processes may include various fluidic components in the fluidic subsystem, various optical components in the optic subsystem, as well as additional components not illustrated in FIG. 1 that may include microprocessor and/or microcontroller components for local control of some functions. In some embodiments samples may be optionally prepared for sequencing in an automated or partially automated fashion using sample preparation instrument 180 configured to perform some or all of the necessary preparation for sequencing using instrument 100. Further, as illustrated in FIG. 1 sequencing instrument 100 may be operatively linked to one or more external computer components such as computer 130 that may for instance execute system software or firmware such as application 135 that may provide instructional control of one or more of the instruments such as sequencing instrument 100 or sample preparation instrument 180, and/or data analysis functions. Computer 130 may be additionally operatively connected to other computers or servers via network 150 that may enable remote operation of instrument systems and the export of large amounts of data to systems capable of storage and processing. In the present example, sequencing instrument 100 and/or computer 130 may include some or all of the components and characteristics of the embodiments generally described above.

In general, embodiments of the invention include a two stage PCR technique (i.e. producing first and second amplicons as described above) targeted to specific regions of HLA, coupled with a sequencing technique that produces sequence information from thousands of nucleic acid molecules in parallel which enables identification of the frequency of occurrence of HLA types present, even those types occurring at a very low frequency in a sample. It will be appreciated that in typical HLA typing embodiments the HLA type for an individual would be completely homozygous where the type would be detected at about a 100% frequency or completely heterozygous where each type would be detected at about 50% frequency. However, embodiments of the invention can detect HLA types present in a sample containing HLA in non-stoichiometric allele amounts, such as, for example, HLA types present at greater than 50%, less than 50%, less than 25%, less than 10%, less than 5% or less than 1%. For example, for a sample derived from a single individual using specific amplification one would expect to detect 100% or 50% (in a heterozygote) of an HLA allele. However one might detect, for example, 5% or 10%, in a complex mixture derived from more than one individual, such as a forensics specimen with multiple contributors (blood from suspect and victim) or in a blood sample monitoring engraftment following a bone marrow transplant (mixture of donor and recipient) or in the SCIDS example with 1-2% maternal cells. The described embodiments enable such identification in a rapid, reliable, and cost effective manner.

In the described embodiments the second round of amplification typically occurs using the emulsion based PCR amplification strategy described above that results in the immobilized clonal population of “second” amplicons on a bead substrate that effectively sequesters the second amplicons preventing diffusion when the emulsion is broken. Typically, thousands of the second amplicons are then sequenced in parallel as described elsewhere in this specification. For example, beads with immobilized populations of second amplicons may be loaded onto reaction substrate 105 and processed using sequencing instrument 100 which generates >1000 clonal reads from each sample and outputs the sequence data to computer 130 for processing. Computer 130 executes specialized software (such as for instance application 135) to identify the HLA type(s) for the loci on interest present in the sample.

As described above, sequencing many nucleic acid templates in parallel provides the sensitivity for the presently described invention as described above. For example, based on binomial statistics the lower limit of detection (i.e., one event) for a fully loaded 60 mm.times.60 mm PicoTiterPlate (2×10⁶ high quality bases, comprised of 200,000×100 base reads) with 95% confidence, is for a population with allelic frequency of at least 0.002%, and with 99% confidence for a population with allelic frequency of at least 0.003% (it will also be appreciated that a 70×75 mm PicoTiterPlate could be employed as described above, which allows for an even greater number of reads and thus increased sensitivity). For comparison, SNP detection via pyrophosphate based sequencing has reported detection of separate allelic states on a tetraploid genome, so long as the least frequent allele is present in 10% or more of the population (Rickert et al., 2002 BioTechniques. 32:592-603). Conventional fluorescent DNA sequencing is even less sensitive, experiencing trouble resolving 50/50 (i.e., 50%) heterozygote alleles (Ahmadian et al., 2000 Anal. BioChem. 280:103-110).

For the purposes of example, Table 1 shows the probability of detecting zero, or one or more, events, based on the incidence of SNP's in the total population, for a given number N (=100) of sequenced amplicons. “*” indicates a probability of 3.7% of failing to detect at least one event when the incidence is 5.0%; similarly, “**” reveals a probability of 0.6% of failing to detect one or more events when the incidence is 7%.

The table thus indicates that the confidence level to detect a SNP present at the 5% level is 95% or better and, similarly, the confidence of detecting a SNP present at the 7% level is 99% or better

TABLE 1

| | Prob. of at least 1 event | Prob. of no event |
|---|---|---|
| Incidence (%) | (N = 100) | (N = 100) |
| 1 | 0.264 | 0.736 |
| 2 | 0.597 | 0.403 |
| 3 | 0.805 | 0.195 |
| 4 | 0.913 | 0.087 |
| 5 | 0.963 | 0.037 * |
| 6 | 0.985 | 0.015 |
| 7 | 0.994 | 0.006 ** |
| 8 | 0.998 | 0.002 |
| 9 | 0.999 | 0.001 |
| 10 | 1.000 | 0.000 |

Naturally, multiplex analysis is of greater applicability than depth of detection and Table 2 displays the number of SNPs that can be screened simultaneously on a single PicoTiterPlate array, with the minimum allelic frequencies detectable at 95% and 99% confidence.

TABLE 2

| | | Minimum | Minimum |
|---|---|---|---|
| | | frequency of SNP in | frequency of SNP in |
| | | population | population |
| SNP | Number of | detectable with 95% | detectable with 99% |
| Classes | Reads | confidence | confidence |
| 1 | 200000 | 0.002% | 0.003% |
| 2 | 100000 | 0.005% | 0.007% |
| 5 | 40000 | 0.014% | 0.018% |
| 10 | 20000 | 0.028% | 0.037% |
| 50 | 4000 | 0.14% | 0.18% |
| 100 | 2000 | 0.28% | 0.37% |
| 200 | 1000 | 0.55% | 0.74% |
| 500 | 400 | 1.39% | 1.85% |
| 1000 | 200 | 2.76% | 3.64% |

Embodiments of the described invention provide methods of HLA genotyping based on the discovery that a multiplex, parallel clonal sequencing analysis can be used to genotype at least 3, typically at least 6, and preferably at least 8 HLA loci in multiple individuals at the same time. The sequencing platforms described herein clonally propagate in parallel millions of single DNA molecules which are then also sequenced in parallel. It will be appreciated that the read lengths obtainable by the described sequencing platforms (i.e. GS FLX or GS Junior sequencing platforms available from 454 Life Sciences Corporation) are typically >500 nucleotides. These clonal read lengths make possible setting the phase of the linked polymorphisms within an exon and thus the unambiguous determination of the sequence of each HLA allele. It is important to note that the described sequencing technologies with read lengths of 500 bases or more enable the acquisition of the complete sequence composition for the loci of interest as a single read in both directions. For example, each strand of the double stranded DNA for the region comprising one or more loci may be simultaneously sequenced in the 5′-3′ direction producing a complete read across said loci enabling unambiguous HLA typing. Thus a higher level of confidence is achieved due to the fact that each nucleotide position in the loci of interest has been interrogated and reviewed in both the forward and reverse directions.

In the described invention, the system is sufficiently high throughput to enable a complete, 8-locus HLA typing for multiple individuals, e.g., 24, 48, or more subjects, in a single sequencing run using a next-generation sequencing platform as described herein. The highly multiplexed amplicon sequencing of the described embodiments employ sample-specific internal sequence tags (i.e. MIDs as described above) in the primers that allow pooling of samples yet maintain the ability to assign sequences to a specific individual. In the described embodiments, the HLA genotypes for at least eight loci (HLA-A, B, C, DRB1, DQA1, DQB1, DPA1, DPB1), as well as for DRB 3, 4, and 5 can be obtained from the data generated by sequencing. This HLA sequencing system can also detect chimeric mixtures, e.g., the detection of the rare non-transmitted maternal allele present in the blood of SCID patients as referenced above. For example, those of ordinary skill in the related art appreciate that SCID (also sometimes referred to as “Bubble Boy Disease”) can include the presence of a third allele in cells of maternal origin in circulation within an individual. The individuals containing cells with the non-transmitted maternal alleles (i.e. maternal cells) are sometimes referred to as “Micro Chimeras” and the maternal cells typically occur at a very low frequency (i.e., about 1-2%) yet have a profound effect upon the individual who often lacks a functional immune system.

Those of ordinary skill in the related art appreciate that the human leukocyte antigen system (HLA) complex spans approximately 3.5 million base pairs on the short arm of chromosome 6. The major regions are the class I and class II regions. The major Class I antigens are HLA-A, HLA-B, and HLA-C and the major Class II antigens are HLA-DP, HLA-DQ and HLA-DR. The HLA-DP, HLA-DQ and HLA-DR loci encode the alpha and beta chains of the HLA-DR, DP and DQ antigens. The HLA genes are among the most polymorphic genes in the genome. Polymorphisms that are expressed in the HLA antigen (and therefore of great interest for typing for transplantation) are localized primarily in exon 2 for the class II genes and exons 2 and 3 for the class I genes. In the presently described embodiments, the read lengths attainable employing the HLA primers and sequencing system described herein enable complete sequencing through the HLA regions important for accurate typing including exon 2 and exon 3. For example, those of ordinary skill in the related art will appreciate that in most individuals HLA-A*01010101 typically comprises about 73 sequence positions in exon 1, about 130 sequence positions in intron 1, about 270 sequence positions in exon 2, about 241 sequence positions in intron 2, about 276 sequence positions in exon 3, about 578 sequence positions in intron 3, about 276 sequence positions in exon 4, about 102 sequence positions in intron 4, about 117 sequence positions in exon 5, about 442 sequence positions in intron 5, about 33 sequence positions in exon 6, about 142 sequence positions in intron 6, about 48 sequence positions in exon 7, about 169 sequence positions in intron 7, and about 5 sequence positions in exon 8.

In embodiments of the described invention, the genotype of an HLA gene as described herein refers to determining the variations in HLA type (which include various polymorphisms) present in the HLA loci. For HLA-A, the variants present in exon 2 and exon 3 are determined by sequencing the products of first amplicons generated by PCR from an individual. In typical embodiments, the sequence of exon 4 is also determined. Exon 2, exon 3, and exon 4, or regions thereof that comprise the allelic determinants, are each amplified in individual PCR reactions to obtain first amplicons. Similarly, first amplicons are obtained for exon 2 and exon 3, and in some embodiments, exon 4, for the HLA-B and HLA-C alleles for an individual. For genotyping HLA class II alleles, first amplicons are obtained for exon 2 of DRB1, DPB1, DPA1, DQA1 and exons 2 and 3 of DQB1. Each exon can be sequenced completely by sequencing the products of first amplicons generated from both strands with sufficient overlap between the reads from either end that specific HLA alleles can be unambiguously assigned.

When allelic determinants are targeted within an exon the amplification primers can be placed fully or partly within that exon, neighboring exons, or more distant exons within the locus, or partly or entirely within the introns or 5′ untranslated region of the locus, or any combination thereof. For example, determinants in HLA-C exon 6 are captured by an amplicon whose primers lie in intron 5 and in intron 7. In some cases allelic determinants in the 5′ untranslated region or introns are also of interest since, for example, certain null alleles may be formed by variations at the splice junctions (either on the exon or intronic side of the junction). Similarly, primers to capture this information by amplification can be placed fully or partly in the 5′ untranslated region, exons and introns of the locus in any combination. For example, information in the HLA-C 5′ untranslated region is captured in an amplicon with primers in the 5′ UTR and in exon 2.

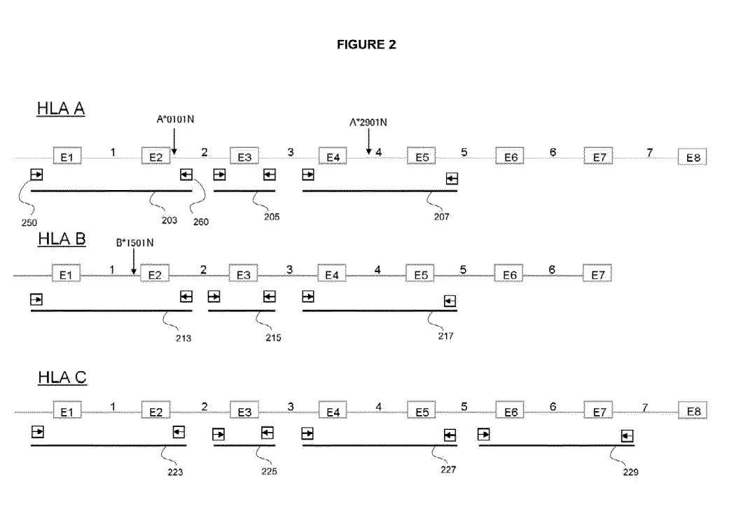

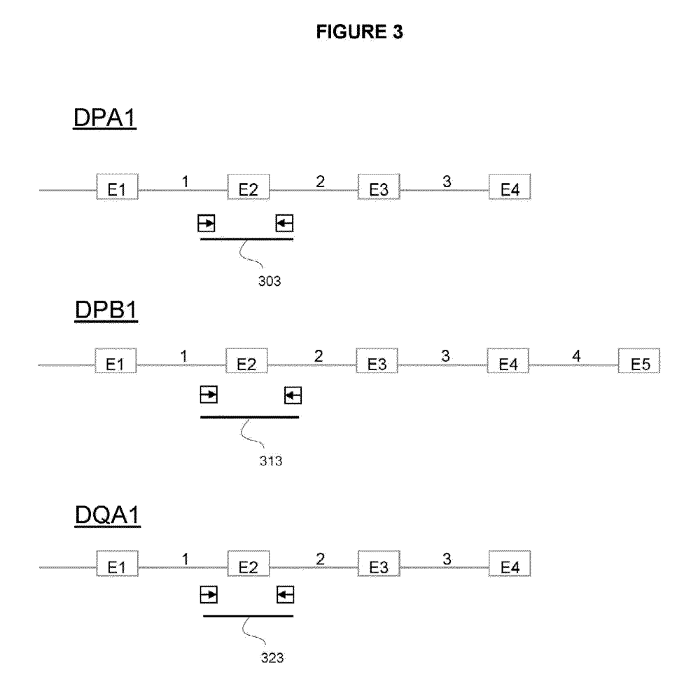

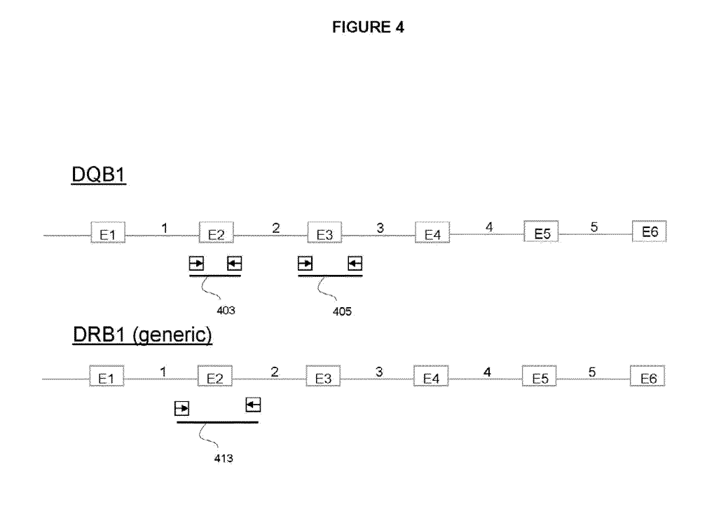

FIGS. 2-4 provide a simplified graphical example of the relationship between the first amplicons generated in embodiments of the invention to the respective HLA region. For instance, FIG. 2 illustrates first amplicon 203 that spans a region comprising exon 1, intron 1, and exon 2; first amplicon 205 that spans a region comprising exon 3; and first amplicon 207 that spans a region comprising exon 4, intron 4, and exon 5 of the HLA-A allele using HLA specific forward primer 250 and reverse primer 260. FIG. 2, also illustrates similar relationships for first amplicons 213, 215, and 217 of the HLA-B allele; and first amplicons 223, 225, and 227 of the HLA-C allele with an additional first amplicon 229 that spans a region comprising exon 6, intron 6, and exon 7. Similarly, FIG. 3 illustrates first amplicons 303, 313, and 323 that span a region comprising exon 2 of the DPA1, DPB1, and DQA1 alleles respectively; and FIG. 4 illustrates first amplicons 403, and 413 that span a region comprising exon 2 of the DQB1, and DRB1 alleles with the addition of first amplicon 405 that spans a region comprising exon 3 of the DRB 1 allele. It will be appreciated that the graphical representations provided in FIGS. 2-4 are for the purposes of illustration and should not be considered limiting.

Each sample from an individual is amplified at one or more loci individually using primers that target the loci of interest that typically include a polymorphic region of one or more exons of interest. The primers employed in the amplification reaction may include additional sequence element such as adapter sequences for emulsion PCR and an identifying MID sequence element that serves as a marker for the DNA from a single individual.

The invention employs amplification primers that amplify the loci of interest of the HLA genes. Typically, the primers are designed to ensure that the entire polymorphic portion of an exon is obtained.

In the described embodiments, primer sequences for the multiplex amplification of the invention are incorporated into adaptors that include sequence elements that can be used to facilitate the clonal sequencing and the analysis. The adaptors of some or all of the described embodiments therefore include the following components: a general adaptor element, a unique identification (i.e. MID) tag and a primer sequence that hybridizes to an HLA gene of interest to use in an amplification reaction to obtain a first HLA amplicon. For example, a schematic representation of an adaptor may include:

General Adapter Sequence+MID Sequence+Target-specific Sequence

The general adaptor elements of the described embodiments may comprise various sequence elements and are typically present at the 5′ end of the adaptors. For example, the general adapter regions may comprise sequences that serve as the site of annealing of primers for the sequencing reaction and also correspond to sequences present on beads, or a solid surface, so that the first amplicon can be annealed to the surface for emulsion PCR. The forward primer for amplifying an HLA exon includes an adapter sequence at the 5′ end, referred to here as the adapter region A. The reverse primer comprises a region that contains an adapter sequence at the 5′ end, referred to here as adapter region B. As noted, the sequences present in the adaptor region and their complements allow for annealing of the first amplicons to beads for emulsion PCR as well as the populations of second amplicons which result from the emPCR process. Optionally, the adaptor may further include a unique discriminating key sequence comprised of a non-repeating nucleotide sequence (i.e., ACGT, CAGT, etc.). This key sequence is typically incorporated to bioinformatically distinguish the sequenced populations of second amplicons for HLA genotyping from control sequences that are included in the reaction.

In the described embodiments the general adaptor sequence may include the following sequences:

It will be appreciated that the described invention is not limited to the exact composition of the general adaptor sequences described above and that different sequence compositions may be used.

PCR primers for use in the described embodiments of HLA genotyping method further comprise MID sequence elements as described above. These MID sequence elements are used to bioinformatically distinguish the sequenced HLA second amplicons from each individual tested. In the described embodiments, HLA regions of interest are amplified from a nucleic acid sample from a subject to be genotyped. For example, the HLA exons, or regions of the exons, comprising the variants that act as allelic determinants are individually amplified. The first amplicons obtained from the subject are marked with the same MID sequence element associating the first amplicons with the subject. In the present example, the MID sequence element is included in the adaptors that are used to amplify each first amplicon for that subject as well as subsequent amplification producing the populations of second amplicons. Accordingly, the MID sequence elements are also sequenced in the sequencing reaction and the sequence composition of each first amplicon (i.e. via sequencing of the respective population of second amplicons) are bioinformatically deconvoluted to associate the sequence composition, and variants contained therein, with the subject.

Table 3 provides examples of MID sequence element useable with embodiments of the described invention.

TABLE 3

| | | | | | 10 BP MID's | | | | | | | |
|---|---|---|---|---|---|---|---|---|---|---|---|---|
| | | | | | | SEQ | | SEQ | | SEQ | | SEQ |
| 4 BP | 5 BP MID's | | | | | ID | | ID | | ID | | ID |
| MID's | 5′T | 5′C | 5′A | 5′G | 5′T | NO: | 5′C | NO: | 5′A | NO: | 5′G | NO: |
| TCAG | TCAGC | TCAGA | TCAGC | TCAGA | ACGCTCGACA | 5 | ACGAGTGCGT | 17 | ACGAGTGCGT | 17 | ACGAGTGCGT | 17 |
| TCAT | TCATC | TCATG | TCATG | TCATC | AGACGCACTC | 6 | ACGCTCGACA | 5 | AGACGCACTC | 6 | ACGCTCGACA | 5 |
| TCTC | TCTCA | TCTCT | TCTCT | TCTCA | AGCACTGTAG | 7 | AGCACTGTAG | 7 | AGCACTGTAG | 7 | AGACGCACTC | 6 |
| TCTG | TCTGC | TCTGA | TCTGC | TCTGA | ATCAGACACG | 8 | ATCAGACACG | 8 | ATCAGACACG | 8 | CGTGTCTCTA | 10 |
| TGAT | TGATC | TGATG | TGATG | TGATC | ATATCGCGAG | 9 | ATATCGCGAG | 9 | ATATCGCGAG | 9 | CTCGCGTGTC | 11 |
| TGAG | TGAGC | TGAGA | TGAGC | TGAGA | CGTGTCTCTA | 10 | CGTGTCTCTA | 10 | CTCGCGTGTC | 11 | TAGTATCAGC | 12 |
| TGCT | TGCTC | TGCTG | TGCTG | TGCTC | CTCGCGTGTC | 11 | TCTCTATGCG | 13 | TAGTATCAGC | 12 | TGATACGTCT | 18 |
| TCCA | TGCAG | TGCAT | TGCAG | TGCAT | TAGTATCAGC | 12 | TGATACGTCT | 18 | TCTCTATGCG | 13 | TACTGAGCTA | 14 |
| CAGA | CAGAG | CAGAT | CAGAG | CAGAT | TCTCTATGCG | 13 | TACTGAGCTA | 14 | TGATACGTCT | 18 | CGAGAGATAC | 16 |
| CAGC | CAGCA | CAGCT | CAGCT | CAGCA | TACTGAGCTA | 14 | CATAGTAGTG | 15 | CATAGTAGTG | 15 | CATAGTAGTA | 20 |
| CATC | CATCA | CATCT | CATCT | CATCA | CATAGTAGTG | 15 | CGTGTCTCTG | 19 | CGAGAGATAC | 16 | ACGAGTGCGA | 21 |
| CATG | CATGC | CATGA | CATGC | CATGA | CGAGAGATAC | 16 | CATAGTAGTA | 20 | CGTGTCTCTG | 19 | CATAGTAGTC | 22 |
| CTCT | CTCTC | CTCTG | CTCTG | CTCTC | | | | | | | | |
| CTCA | CTCAG | CTCAT | CTCAG | CTCAT | | | | | | | | |
| CTGA | CTGAG | CTGAT | CTGAG | CTGAT | | | | | | | | |
| CTGC | CTGCA | CTGCT | CTGCT | CTGCA | | | | | | | | |
| ATCA | ATCAG | ATCAT | ATCAG | ATCAT | | | | | | | | |
| ATCT | ATCTC | ATCTG | ATCTG | ATCTC | | | | | | | | |
| ATGA | ATGAG | ATGAT | ATGAG | ATGAT | | | | | | | | |
| ATGC | ATGCA | ATGCT | ATGCT | ATGCA | | | | | | | | |
| AGCA | AGCAG | AGCAT | AGCAG | AGCAT | | | | | | | | |
| AGCT | AGCTC | AGCTG | AGCTG | AGCTC | | | | | | | | |
| AGAT | AGATC | AGATG | AGATG | AGATC | | | | | | | | |
| AGAG | AGAGC | AGAGA | AGAGC | AGAGA | | | | | | | | |

In embodiments of the described invention the MID sequences can be designed taking into account certain parameters which may include some or all of the parameters described above. For example, in designing a 4-residue MID tag, it is desirable to choose 4 bases that take into account the flow cycle of the nucleotides in the sequencing reaction. In the present example, if the nucleotides are added in the order T, A, C, and G, it is typically desirable to design the MID sequence such that a nucleotide that is positive (i.e. nucleotide in the flow is complementary to the next nucleotide in MID sequence) is followed by a residue that would be negative (i.e. nucleotide in the flow is non-complementary to the next nucleotide in MID sequence). Accordingly, in this example, if an MID sequence begins with an “A” nucleotide such that the nucleotide incorporated in the sequencing reaction is T, the second nucleotide in the tag sequence would be a nucleotide such that A would not be incorporated. In addition, it is desirable to avoid forming homopolymers, either within the MID sequence or through creating them based on the last nucleotide of the adapter region or the first nucleotide of the HLA-specific primer region of the adaptor.

The target-specific sequence (also referred to herein as HLA priming region, HLA binding region, or HLA hybridizing region) of the described adaptors is the region of the primer that hybridizes to the HLA sequence of interest to amplify the desired locus that may include an exon, combination of two exons and intervening intron sequence, or in some embodiments, a limited region of the exon. Typically, the HLA priming region of the adaptor hybridizes to intronic sequence adjacent to the exon to be amplified in order to obtain the entire exon sequence. The HLA primer sequences are preferably selected to selectively amplify the HLA exon of interest, although in some embodiments, a primer pair may also amplify a highly similar region of a related region of HLA gene. For example, the primers for exon 2 of DRB1 described in the example section below also amplify the DRB3, DRB4, and DRB5 loci (i.e. they are “generic” to those loci). The primer sequences are selected such that the exon is amplified with sufficient specificity to allow unambiguous determination of the HLA genotype from the sequence.

Consensus sequences of HLA genes and alleles are known and available through various databases, including GenBank and other gene databases and have been published (see e.g., Mason and Parham (1998) Tissue Antigens 51: 417-66, listing HLA-A, HLA-B, and HLA-C alleles; Marsh et al. (1992) Hum. Immunol. 35:1, listing HLA Class II alleles—DRA, DRB, DQA1, DQB1, DPA1, and DPB1).

The PCR primers were designed based on principles known in the art. Strategies for primer design may be found throughout the scientific literature, for example, in Rubin, E. and A. A. Levy, Nucleic Acids Res, 1996.24 (18): p. 3538-45; and Buck et al., Biotechniques, 1999.27 (3): p. 528-36. For example, the HLA-specific primer is typically about 20 nucleotides or greater, e.g., 20 to 35 nucleotides in length. Other parameters that are considered are G/C content, design considerations to avoid internal secondary structure, and prevent the formation of primer dimers, as well as melting temperatures (Tm).

Examples of HLA target specific primers for use in embodiments of the invention are provided in Table 4.

TABLE 4
HLA Target-Specific Primer Sequences

| | SEQ ID | |
|---|---|---|
| | No: | |
| HLA-A | | |
| HLA-A Exon 2 5′ | 23 | GAAACGGCCTCTGTGGGGAGAAGCAA |
| HLA-A Exon 1-2 3′ | 24 | GGTGGATCTCGGACCCGGAGACTGT |
| HLA-A Exon 3 5′ | 25 | GACTGGGCTGACCGTGGGGT |
| HLA-A Exon 3 3′ | 26 | CCCCTGGTACCVGTGCGCTGCA |
| HLA-A Exon 3 5′ | 27 | GACTGGGCTGACCGTGGGGT |
| HLA-A Exon 3 3′ | 28 | GAGGGTGATATTCTAGTGTTGGTCCCAA |
| HLA-A Exon 4 5′ | 29 | TGCCTGAATGWTCTGACTCTTCCCGTMAGA |
| HLA-A Exon 4 3′ | 30 | TGACCCTGCTAAAGGTCTCCAGAG |
| HLA-A Exon 4 3′ | 30 | TGACCCTGCTAAAGGTCTCCAGAG |
| HLA-A Exon 4 5′ | 31 | CTGGGTTCTGTGCTCYCTTCCCCAT |
| HLA-A Exon 4 3′ | 32 | CTCCAGAGAGGCTCCTGCTTTCCSTA |
| HLA-B | | |
| HLA-B Exon 2 5′ | 33 | AGAGCTCGGGAGGAGCGAGGGGACCSCAG |
| HLA-B Exon 2 3′ | 34 | ACTCGAGGCCTCGCTCTGGTTGTAGTA |
| HLA-B Exon 2 3′ | 35 | CGGTCGAGGGTYTGGGC |
| HLA-B Exon 3 5′ | 36 | AGAGCTCGGGCCAGGGTCTCACA |
| HLA-B Exon 3 3′ | 37 | ACTCGAGGGAGGCCATCCCCGGCGACCTAT |
| HLA-B Exon 3 5′ | 38 | CCCGGTTTCATTTTCAGTTGAGG |
| HLA-B Exon 4 5′ | 39 | GCGCCTGAATTTTCTGACTCTTCCCA |
| HLA-B Exon 4 3′ | 40 | GGCTCCTGCTTTCCCTGAGAA |
| HLA-B Exon 4 5′ | 41 | CTGGTCACATGGGTGGTCC |
| HLA-B Exon 4 3′ | 42 | AGATATGACCCCTCATCCC |
| HLA-C | | |
| HLA-C Exon 2 5′ | 43 | AGTCGACGAADCGGCCTCTGSGGA |
| HLA-C Exon 2 3′ | 44 | ACTCGAGGGGCYGGGGTCACTCAC |
| HLA-C Exon 3 5′ | 45 | ACGTCGACGGGCCAGGKTCTCACA |
| HLA-C Exon 3 3′ | 46 | ACCTCGAGGTCAGCAGCCTGACCACA |
| HLA-C Exon 3 3′ | 47 | CTCCCCACTGCCCCTGGTAC |
| nested | | |
| HLA-C Exon 4 5′ | 48 | CAAAGTGTCTGAATTTTCTGACTCTTCCC |
| HLA-C Exon 4 3′ | 49 | TGAAGGGCTCCAGAAGGACTT |
| HLA-C Exon 4 3′ | 50 | TGAAGGGCTCCAGGACTT |
| HLA-C Exon 4 5′ | 51 | GTGTCGCAAGAGAGATRCAAAGTGT |
| HLA-C Exon 4 3′ | 52 | GAGGRGAAGGTGAGGGGCC |
| DPB1 | | |
| DPB1 Exon 2 5′ | 53 | GCTGCAGGAGAGTGGCGCCTCCGCTCAT |
| DPB1 Exon 2 3′ | 54 | CGGATCCGGCCCAAAGCCCTCACTC |
| DQ | | |
| DQA1 Exon 2 5′ | 55 | GTTTCTTYCATCATTTTGTGTATTAAGGT |
| DQA1 Exon 2 3′ | 56 | CGGTAGAGTTGTAGCGTTTA |
| DQA1 Exon 2 5′ | 57 | GTCAGTTTCTTYCATCATTTTGTGTATTAAGGT |
| DQA1 Exon 2 5′ | 58 | GAAAGTCAGTTTCTTYCATCATTTTGTGTATTAA |
| DQA1 Exon 2 3′ | 59 | CCATGASAAGATCTGGGGACCTCT |
| DQA1 Exon 2 3′ | 56 | CGGTAGAGTTGTAGCGTTTA |
| DQB1 Exon 2 5′ | 60 | AGGATCCCCGCAGAGGATTTCGTGTACCA |
| DQB1 Exon 2 3′ | 61 | TCCTGCAGGACGCTCACCTCTCCGCTGCA |
| DQB1 Exon 3 5′ | 62 | TGGAGCCCACAGTGACCATCTCC |
| DQB1 Exon 3 3′ | 63 | GCTGGGGTGCTCCACGTGGCA |
| DQB1 Exon 3 5′ | 62 | TGGAGCCCACAGTGACCATCTCC |
| DQB1 Exon 3 3′ | 64 | AGTGACATCAGGGATAAGAGATGGGAA |
| DRB1 | | |
| DRB1 generic 5′ | 65 | CCGGATCCTTCGTGTCCCCACAGCACG |
| DRB1 generic 3′ | 66 | CCGAATTCCGCTGCACTGTGAAGCTCTC |
| DRB1 generic 5′ | 67 | CCGGATCCTTCGTGTCCCCACAG |
| DRB1 generic 3′ | 68 | GATTCTRAATGCTCACAGATGGCG |

Further Table 5 provides additional examples of HLA target specific primers useable for embodiments of the described invention. Those of ordinary skill in the related art will appreciate that the target specific primer sequences in Tables 4 and 5 may be used interchangeably with one another for the same target loci. It will also be noted that some of the HLA specific primer sequences in the adaptor sequences of Table 5 may be the same as those in Table 4 however for some HLA loci some differences exist.

TABLE 5
HLA adaptors

| | | | | SEQ |
|---|---|---|---|---|
| | | | | Id |
| Locus | | Name | Sequence | No: |
| A1-2 | 5′ | PM1283 | GTTTCCAGAGAAGCCAATCAGTGTCGT | 69 |
| A1-2 | 5′ | PM1277 | TAAAGTCCGCACGCACCCACCG | 70 |
| A4-5 | 3′ | PM1280 | CTTGGAACCCTCAGTGAGACAAGAAAT | 71 |
| A4-5 | 3′ | PM1281 | TTGGAACCCTCAGTGAGACAAGAAAT | 72 |
| A4-5 | 3′ | PM1282 | CTGGGGCTTGGAACCCTCAGTGA | 73 |
| A4-5 | 5′ | PM1288 | GGTTCTGTGCTCYCTTCCCCAT | 74 |
| A4-5 | 3′ | PM1289 | GGAACCCTCAGTGAGACAAGAAAT | 75 |
| A4-5 | 3′ | PM1290 | GGGCTTGGAACCCTCAGTGA | 76 |
| B1-2 | 5′ | FHLAB1-2TV1 | GCACCCACCCGGACTCAGARTCTCCT | 77 |
| B1-2 | 5′ | FHLAB1-2TV2 | CCACCCGGACTCAGARTCTCCT | 78 |
| B1-2 | 3′ | RHLAB1-2TV1 | CCGGGCCGGGGTCACTCAC | 79 |
| B1-2 | 3′ | RHLAB1-2TV2 | GGGCCGGGGTCACTCAC | 80 |
| B1-2 | 3′ | RHLAB1-2TV3 | CCCGCGGGGATTTTGGCCTC | 81 |
| B1-2 | 3′ | RHLAB1-2TV4 | CGCGGGGATTTTGGCCTC | 82 |
| B3 | 5′ | FHLAB3TV1 | CGCGTTTACCCGGTTTCATTTTCAGTTG | 83 |
| B3 | 5′ | FHLAB3TV2 | CGTTTACCCGGTTTCATTTTCAGTTG | 84 |
| B3 | 5′ | FHLAB3TV3 | CCCGGTTTCATTTTCAGTTGAGGYCAA | 85 |
| B3 | 5′ | FHLAB3TV4 | GGTTTCATTTTCAGTTGAGGYCAA | 86 |
| B3 | 3′ | RHLABC3TV1 | GGAGATGGGGAAGGCTCCCCACT | 87 |
| B3 | 3′ | RHLABC3TV2 | ATGGGGAAGGCTCCCCACT | 88 |
| B3 | 3′ | RHLABC3TV3 | AGGGGGCCCTCAGAGGAAACT | 89 |
| ABC3 | 5′ | FCLASS13TV1 | GTTTAGGCCAAAATCCCCGCGG | 90 |
| B4-5 | 5′ | FHLAB4-5TV1 | AAAGCGCCTGAATTTTCTGACTCTTCCCA | 91 |
| B4-5 | 5′ | FHLAB4-5TV2 | CGCCTGAATTTTCTGACTCTTCCCA | 92 |
| B4-5 | 3′ | RHLAB4-5TV1 | GCTGCTTCCCAGTAATGAGGCAGGGA | 93 |
| B4-5 | 3′ | RHLAB4-5TV2 | GCTTCCCAGTAATGAGGCAGGGA | 94 |
| B4-5 | 3′ | RHLAB4-5TV3 | TGCGTTAGCCCCTGTGTGSATGC | 95 |
| B4-5 | 3′ | RHLAB4-5TV4 | CGTTAGCCCCTGTGTGSATGC | 96 |
| C1-2 | 5′ | FHLAC1-2TV1 | CGGGTTCTAGAGAAGCCAATCAGCGTCT | 97 |
| C1-2 | 5′ | FHLAC1-2TV2 | GGTTCTAGAGAAGCCAATCAGCGTCT | 98 |
| C1-2 | 5′ | FHLAC1- | TTCTAGAGAAGCCAATCAGCGTCT | 99 |
| | | 2TV3_1 | | |
| C1-2 | 3′ | RHLAC1-2TV1 | GGTCGAGGGTCTGGGCGGGTT | 100 |
| C1-2 | 3′ | RHLAC1-2TV2 | CGAGGGTCTGGGCGGGTT | 101 |
| C1-2 | 3′ | RHLAC1- | CCGGGCYGGGGTCACTCAC | 102 |
| | | 2TV3_1 | | |
| C3 | 5′ | FHLAC3TV1 | CGCCCAGACCCTCGACCGGA | 103 |
| C3 | 5′ | FHLAC3TV2 | CCCAGACCCTCGACCGGA | 104 |
| C3 | 3′ | RHLAC3TV4_1 | GAGAGAAAGGTCAGCAGCCTGACCACA | 105 |
| C3 | 3′ | RHLAC3TV5_1 | AAAGGTCAGCAGCCTGACCACA | 106 |
| C3 | 5′ | FHLAC3TV3_1 | CCTCGACCGGAGAGAGCCCYAGT | 107 |
| C3 | 5′ | FHLAC3TV4_1 | CGACCGGAGAGAGCCCYAGT | 108 |
| C4-5 | 5′ | FHLAC4-5TV1 | TCCATTCTCAGGATGGTCACATGGGC | 109 |
| C4-5 | 5′ | FHLAC4-5TV2 | ATTCTCAGGATGGTCACATGGGC | 110 |
| C4-5 | 3′ | RHLAC4-5TV1 | GGGCACACTTCTACCTGGGGCTTGAAACT | 111 |
| C4-5 | 3′ | RHLAC4-5TV2 | CACACTTCTACCTGGGGCTTGAAACT | 112 |
| C4-5 | 3′ | RHLAC4-5TV3 | CACACAGGGTCCCAGGCTGGGA | 113 |
| C4-5 | 3′ | RHLAC4-5TV4 | ACAGGGTCCCAGGCTGGGA | 114 |
| C6-7 | 5′ | FHLAC6-7TV1 | ACTTCTCTTGGGTCCAAGACTAGGAGGTTCCC | 115 |
| C6-7 | 5′ | FHLAC6-7TV2 | TGGGTCCAAGACTAGGAGGTTCCC | 116 |
| C6-7 | 3′ | RHLAC6-7TV1 | CCCACCCCCGACCACTTCAGCT | 117 |
| C6-7 | 3′ | RHLAC6-7TV2 | CACCCCCGACCACTTCAGCT | 118 |
| C6-7 | 3′ | RHLAC6-7TV3 | GAAACGTCCCAATCAAAGRATCCCCATTA | 119 |
| C6-7 | 3′ | RHLAC6-7TV4 | CGTCCCAATCAAAGRATCCCCATTA | 120 |
| DPA1 | 5′ | PM1272 | GACCACTTGCATATTCAAACTGA | 121 |
| DPA1 | 3′ | PM1274 | GGCTACAGAGGAAGAGGCAAAGATA | 122 |
| DPA1 | 5′ | PM1273 | GACCACTTGCATATTCAAACTGACA | 123 |
| DPA1 | 3′ | PM059 | GGCTACAGAGGAAGAGGCAAAGATAGG | 124 |
| DRB1 | 5′ | PM1283 | GTTTCCAGAGAAGCCAATCAGTGTCGT | 69 |
| DRB1 | 5′ | PM1284 | CGGATGCTTTGTGGACCCGCA | 125 |
| DRB1 | 5′ | PM1285 | GGATGCTTTGTGGACCCGCA | 126 |
| DRB1 | 3′ | PM1286 | GGATAGAGAGGATTCTGAATGCTCACAGAT | 127 |
| DRB1 | 3′ | PM1287 | GGATAGAGAGGATTCTGAATGCTCACAGA | 128 |

Those of ordinary skill in the art will appreciate that some variability of sequence composition for primer sets exist and that 90% or greater homology to the disclosed primer sequences are considered within the scope of the presently described invention. For example, the target regions for the sets of primers may be slightly shifted and thus some difference in primer sequence composition is expected. Also, refinements to the consensus sequence may be made or new sequence degeneracy at certain positions may be discovered resulting in a slight difference of sequence composition in the target region, and similarly some variation in primer sequence composition is expected.

The template nucleic acid used to amplify the HLA first amplicon of interest is typically from genomic DNA isolated from a subject to be genotyped. In the current method, more than one subject is HLA genotyped in parallel reactions. In the current invention, at least 12 subjects, and typically at least 16, 20, 24, 30, 36, or 48 subjects are HLA genotyped. The HLA amplicons may be obtained using any type of amplification reaction. In the described embodiments, first amplicons are typically made by PCR using HLA primer pairs as described herein, where it is typically desirable to use a polymerase with a low error rate, e.g., such as a high-fidelity Taq polymerase (Roche Diagnostic Corp., Indianapolis, Ind.).

The PCR conditions can be optimized to determine suitable conditions for obtaining first HLA amplicons from a subject. Each first HLA amplicon may be individually amplified in separate PCR reactions. In some embodiments, the first HLA amplicons for a subject may be obtained in one or more multiplex reactions that comprise primer pairs to amplify individual amplicons.

In the described embodiments, populations of HLA second amplicons are amplified and immobilized on beads via an emulsion PCR process as described above. For example, the first HLA amplicons are, preferably, individually compartmentalized within an aqueous droplet of a water-in-oil emulsion and attached to a single bead compartmentalized within the droplet by annealing a bead bound primer to the first amplicon, via a complementary primer element in the adaptor region. The bead comprises a large number of the primer species complementary to the primer element in the adapter portion. In the present example, the discrete aqueous phase microdroplets, are approximately 60 to 200 μm in diameter, enclosed by a thermostable oil phase where the emulsion droplets are formed such that on average, the emulsion comprises only one target nucleic acid and one bead. Each microdroplet contains, preferably, amplification reaction solution (i.e., the reagents necessary for nucleic acid amplification, such as polymerase, salts, and appropriate primers, e.g., corresponding to the adaptor region).

In the described embodiments, emulsion PCR is typically performed with two populations of beads, as the first HLA amplicons are sequenced in both directions. In one population of beads, a first primer complementary to the “reverse” primer element in the adapter sequence (i.e. the “B” adaptor) is attached to a bead. In the second population of beads, a second primer complementary to the “forward” primer element in the adapter sequence (i.e. the “A” adaptor) is attached to a bead. Thus, a primer for use in the emulsion amplification reaction typically has the sequence of the adapter region, without additional sequences such as “key” sequences. In some embodiments, the emulsion amplification reaction may be performed with asymmetric primer concentrations in the aqueous solution (i.e. typically the primer species immobilized on the bead will have the lower concentration in solution). For example, the PCR primers may be present in an 8:1 or 16:1 ratio (i.e., 8 or 16 of one primer to 1 of the second primer) to perform asymmetric PCR. However it will be appreciated that the asymmetric primer concentrations may not be necessary and equal primer concentrations may instead be employed in the aqueous solution, or in some preferred embodiments the primer species immobilized on the bead will not be present in the aqueous solution (i.e. the B primer species is immobilized and the A primer species is in solution).

Following emulsion PCR amplification, the beads that have the singled-stranded second HLA amplicon template are isolated, e.g., via a moiety such as a biotin that is present on an amplification primer during the emulsion PCR, and the template is sequenced using DNA sequencing technology described elsewhere in this specification. For example, clonal second amplicons are sequenced using a sequencing primer (e.g., primer A or primer B) and adding four different dNTPs or ddNTPs subjected to a polymerase reaction. As each dNTP or ddNTP is added to the primer extension product, a pyrophosphate molecule is released. Pyrophosphate release can be detected enzymatically, such as, by the generation of light in a luciferase-luciferin reaction. Additionally, a nucleotide degrading enzyme, such as apyrase, can be present during the reaction in order to degrade unincorporated nucleotides. In other embodiments, the reaction can be carried out in the presence of a sequencing primer, polymerase, a nucleotide degrading enzyme, deoxynucleotide triphosphates, and a pyrophosphate detection system comprising ATP sulfurylase and luciferase.

Once the sequencing data is obtained for the sequence of the individual DNA molecules, the unambiguous HLA sequence can be determined by comparing these sequence files to an HLA sequence database for the known HLA alleles. The read lengths achieved by the 454 sequencing system (454 Life Sciences Corporation, Branford, Conn.) (typically at least 500 bp) are sufficient to enable unambiguous determination of the sequence composition of each exon. The assignment of genotypes at each locus based on the exon sequence data files can be performed by application 135. For example, application 135 may include a software application developed by Conexio Genomics. An important aspect of the software is the ability to filter out related sequence reads (pseudogenes and other unwanted HLA genes) that were co-amplified by the primers along with the target sequence. In the same or alternative examples, application 135 may include the Amplicon Variant Analyzer software application (generally referred to as the AVA software) (454 Life Sciences Corporation) that compares the sequence composition generated from each first amplicon against a consensus sequence and identifies all variation that deviates from the consensus. In some embodiments, the AVA software may be additionally enabled to associate variants (or combinations of variation) with variation known type (i.e. HLA type) or variation known to confer a phenotype associated with a disease, condition, resistance, etc. Alternatively, the AVA software may be employed for pre-processing the sequence data where the pre-processed data may subsequently be uploaded into the Conexio software for further processing.

Further, embodiments of the described invention include packaging some or all of the compositions and reagents described herein into kits. A kit of the described embodiments typically comprises multiple adaptor pairs as described herein that are suitable for amplifying the regions of interest in an HLA allele. The adaptor pairs comprise a forward primer comprising a general adapter region, an MID tag and an HLA primer region; and a reverse primer that comprises a general adapter region, an MID tag, and an HLA primer region. It will, however, be appreciated that only one MID tag may be necessary depending on the number of sample associations necessary. The kits of the described embodiments often comprise primer pairs to amplify first amplicons for determining the genotype of multiple subjects for at least HLA-A, HLA-B, and DRB1. Often, a kit of the described embodiments comprise sufficient HLA primer pairs to determine the genotype of HLA-A, HLA-B, HLA-C, DRB1, DQA1, DQB1, DPA1, and DPB1 genes for multiple individuals, e.g., 12 or more individuals.

In some embodiments, a kit can additionally comprise one or more populations of beads that have a primer attached that corresponds to an adapter region that can be used in emulsion PCR. In some embodiments, a kit can comprise one or more reaction compartments comprising reagents suitable for performing a reaction selected at the discretion of a practitioner. For example, in some embodiments, a kit can comprise one or more reaction compartments comprising one more sequencing reagents.

The various components included in the kit are typically contained in separate containers, however, in some embodiments one or more of the components can be present in the same container. Additionally, kits can comprise any combination of the compositions and reagents described herein. In some embodiments, kits can comprise additional reagents that may be necessary or optional for performing the disclosed methods. Such reagents include, but are not limited to, buffers, control polynucleotides, and the like.

The genes of the HLA locus (HLA genes) are the most polymorphic in the human genome. The HLA locus spans approximately 3.5 million base pairs on the short arm of chromosome 6. The major regions are the Class I and Class II regions. The Class I genes are HLA-A, HLA-B, and HLA-C and the major Class II genes are HLA-DP, HLA-DQ and HLA-DR.

HLA typing aims to detect HLA alleles in an individual by testing a sample derived from the individual. The resolution of an HLA typing test refers to the level of discrimination among different HLA genotypes achieved by the test. Specifically, the level of resolution determines how many differences between related alleles are detected. Examples of HLA typing resolution include low, high and allelic resolution as defined e.g., in Nunes, E. et al., (2011) Definitions of histocompatibility typing terms, Blood, 118:e180. Low resolution HLA typing is capable of distinguishing HLA alleles at the protein (antigen) level. High and very high resolution HLA typing are capable of distinguishing HLA alleles at the nucleic acid sequence level. One of skill in the art will recognize that assessing the level of resolution of a particular HLA test may change over time. As more HLA alleles (i.e., different variants of the HLA nucleic acid and protein sequence) are discovered, ever more precise testing will become possible and tests with ever higher levels of resolution will be developed. The resolution may also be defined in terms of a particular downstream application of the HLA test. For example, one may determine the required level of resolution of an HLA test as the level that is sufficient to achieve successful organ or tissue transplantation in among a certain percentage of patients. (see Lee et al., (2007) High resolution donor-recipient HLA matching contributes to the success of unrelated donor marrow transplantation, Blood 110:4576-4583.) Therefore in the context of the present invention, the inventors adopt their own definition of medium resolution (MR), high resolution (HR) and very high resolution (VHR) HLA typing tests as set forth below.

The MR set of HLA loci focuses on the most polymorphic exons 2 and 3 nucleic acid sequences coding for the “peptide binding groove” of Class I genes HLA-A, -B and -C and on exon 2 of the Class II genes DRB1 and DQB1. These are the loci most commonly typed for matching of hematopoietic stem cell transplant donors and recipients (see Lee et al., supra).

The HR set of HLA loci provides additional allele discrimination among Class I genes and DQB1 (some alleles differ only in exon 3), DPB1 and DQA1. The increased resolution has clinical significance for both disease associations and organ and tissue transplantation, e.g., hematopoietic stem cell (HSC) transplantation.

The present invention comprises methods and compositions for very high resolution (VHR) HLA typing that provides additional allele discrimination beyond the capabilities of the high resolution (HR) HLA typing. The invention comprises a unique selection of a set of target HLA loci that comprises the most desirable combination of exons and introns of HLA genes to be typed when considering HLA association with disease (e.g., Type I diabetes, Parkinson's disease, multiple sclerosis) or HLA matching for hematopoietic stem cell (HSC) transplantation. To that end, the invention comprises a set of oligonucleotides that is capable of specifically hybridizing to and directing amplification of the selected HLA loci simultaneously in a single-pass run so that the HLA genotype of an individual may be determined with very high resolution.

The unique properties of the VHR typing that distinguish it from the HR typing include the ability of resolving some additional HLA alleles that are among the Common and Well Documented Alleles as well as the vast majority the commonly encountered “null” alleles, i.e., alleles that are not expressed as proteins due to a nonsense mutation or a mutation in the splice junction. Because the null allele results in a complete absence of a protein, it is important to identify such alleles when matching donors and recipients for hematopoietic stem cell transplantation. Identification of the rare and very rare null alleles is especially important in the final selection of a matched donor during HSC transplantation.

The loci selected for medium (MR) and high resolution (HR) HLA typing are listed in Tables 6 and 7. The oligonucleotides containing HLA-hybridizing regions specific for these loci are disclosed in Table 4. The loci selected for very high resolution (VHR) HLA typing are listed in Table 8. The oligonucleotides used for VHR typing are disclosed in Table 9.

TABLE 6
Loci included in medium resolution (MR) HLA typing

| HLA gene | Exons |
|---|---|
| HLA-A | Exons 2, 3 |
| HLA-B | Exons 2, 3 |
| HLA-C | Exons 2, 3 |
| DQB1 | Exon 2 |
| DRB1 | Exon 2 |
| DRB3 | Exon 2 |
| DRB4 | Exon 2 |
| DRB5 | Exon 2 |

TABLE 7
Loci included in high resolution (HR) HLA typing

| HLA gene | Exons |
|---|---|
| HLA-A | Exons 2, 3, 4 |
| HLA-B | Exons 2, 3, 4 |
| HLA-C | Exons 2, 3, 4 |
| DQB1 | Exon 2, 3 |
| DRB1 | Exon 2 |
| DRB3 | Exon 2 |
| DRB4 | Exon 2 |
| DRB5 | Exon 2 |
| DPB1 | Exon 2 |

TABLE 8
Loci included in very high resolution (VHR) HLA typing

| HLA gene | Exons |
|---|---|
| HLA-A | Exons 1, 2, 3, 4, 5 |
| HLA-B | Exons 1, 2, 3, 4, 5 |
| HLA-C | Exons 1, 2, 3, 4, 5, 6, 7 |
| DQB1 | Exon 2, 3 |
| DRB1 | Exon 2, 3 |
| DRB3 | Exon 2, 3 |
| DRB4 | Exon 2, 3 |
| DRB5 | Exon 2, 3 |
| DPA1 | Exon 2 |
| DPB1 | Exon 2 |

### Sets of Oligonucleotides

In one embodiment, the invention is a set of oligonucleotides specifically hybridizing to and capable of directing amplification of one or more genes and exons selected from HLA-A, Exon 1, 5, HLA-B, Exon 1, 5, HLA-C, Exons 1, 5, 6, 7, DRB1, Exon 3, DRB3, Exon 3, DRB4, Exon 3, DRB5, Exon 3, and DPA1, Exon 2, the set comprising one or more pairs of oligonucleotides disclosed in Table 9. In variations of this embodiment, the invention is a set of oligonucleotides specifically hybridizing to and capable of directing amplification of HLA-A, Exon 1, 5, HLA-B, Exon 1, 5, HLA-C, Exons 1, 5, 6, 7, DRB1, Exon 3, DRB3, Exon 3, DRB4, Exon 3, DRB5, Exon 3, and DPA1, Exon 2, the set comprising the oligonucleotides disclosed in Table 9. In other variations of this embodiment, the invention is a set of oligonucleotides specifically hybridizing to and capable of directing amplification of HLA-A, Exon 1, 5, HLA-B, Exon 1, 5, HLA-C, Exons 1, 5, 6, 7, DRB1, Exon 3, DRB3, Exon 3, DRB4, Exon 3, DRB5, Exon 3, and DPA1, Exon 2, the set consisting of the oligonucleotides disclosed in Table 9.

The set of oligonucleotides of the present invention may be a part of a larger set of oligonucleotides, i.e., the set disclosed herein may be combined with additional oligonucleotides specifically hybridizing to and directing amplification of other HLA sequences. In some embodiments, the invention is a set of oligonucleotides specifically hybridizing to and capable of directing amplification of one or more genes and exons selected from HLA-A, Exons 1, 2, 3, 4, 5, HLA-B, Exons 1, 2, 3, 4, 5, HLA-C, Exons 1, 2, 3, 4, 5, 6, 7, DQB1, Exon 2, 3, DRB1, Exons 2, 3, DRB3, Exons 2, 3, DRB4, Exons 2, 3, DRB5, Exons 2, 3, DPA1, Exon 2, and DPB1, Exon 2, the set being a combination comprising one or more pairs of oligonucleotides disclosed in Table 9 and one or more pairs of oligonucleotides disclosed in Table 4 or Table 5. In variations of this embodiment, the invention is a set of oligonucleotides specifically hybridizing to and capable of directing amplification of HLA-A, Exons 1, 2, 3, 4, 5, HLA-B, Exons 1, 2, 3, 4, 5, HLA-C, Exons 1, 2, 3, 4, 5, 6, 7, DQB1, Exon 2, 3, DRB1, Exons 2, 3, DRB3, Exons 2, 3, DRB4, Exons 2, 3, DRB5, Exons 2, 3, DPA1, Exon 2, and DPB1, Exon 2, the set being a combination comprising the oligonucleotides disclosed in Table 9 and the oligonucleotides disclosed in Table 4 or Table 5. In further variations of this embodiment, the invention is a set of oligonucleotides specifically hybridizing to and capable of directing amplification of HLA-A, Exons 1, 2, 3, 4, 5, HLA-B, Exons 1, 2, 3, 4, 5, HLA-C, Exons 1, 2, 3, 4, 5, 6, 7, DQB1, Exon 2, 3, DRB1, Exons 2, 3, DRB3, Exons 2, 3, DRB4, Exons 2, 3, DRB5, Exons 2, 3, DPA1, Exon 2, and DPB1, Exon 2, the set being a combination consisting of the oligonucleotides disclosed in Table 9 and the oligonucleotides disclosed in Table 4. In further variations of this embodiment, the invention is a set of oligonucleotides specifically hybridizing to and capable of directing amplification of HLA-A, Exons 1, 2, 3, 4, 5, HLA-B, Exons 1, 2, 3, 4, 5, HLA-C, Exons 1, 2, 3, 4, 5, 6, 7, DQB1, Exon 2, 3, DRB1, Exons 2, 3, DRB3, Exons 2, 3, DRB4, Exons 2, 3, DRB5, Exons 2, 3, DPA1, Exon 2, and DPB1, Exon 2, the set being a combination consisting of the oligonucleotides disclosed in Table 9 and the oligonucleotides disclosed in Table 5.

Because HLA genes are both polymorphic and members of multigene families, it is very challenging to design primers that both (1) amplify all allelic variants occurring at a particular locus; and (2) do not amplify related loci in other HLA genes or pseudogenes present in the sample. The ability of the oligonucleotides disclosed herein in combination with e.g., oligonucleotides listed in Tables 4 or Table 5 to perform very high resolution (VHR) HLA genotyping in a set of clinical samples is due to the unique sequence properties of the oligonucleotides. The task of amplifying all alleles at a locus is distinct from the task of amplifying a known gene sequence. In HLA loci, multiple alleles (i.e. multiple differences among the target sequences found in the human population) are present. Each primer might encounter one or more mismatches with the target sequence in the test samples at unpredictable locations. Therefore routine methods of primer design (e.g., those practiced by the VisualOMP™ software, DNA Software, Inc., Ann Arbor, Mich.) are no longer applicable. The inventors discovered that in some instances, designing oligonucleotides with one or more non-natural bases enables successful amplification of all alleles found in the test samples. In other instances, the inventors discovered that designing the oligonucleotide sample as a mixture of two oligonucleotide species differing by one or more base enables successful amplification of all alleles found in the test samples.

For successful extension of a primer, the primer needs to have at least partial complementarity to the target sequence. Generally, complementarity at the 3′-end of the primer is more critical than complementarity at the 5′-end of the primer. (Innis et al. Eds. PCR Protocols, (1990) Academic Press, Chapter 1, pp. 9-11). Therefore the present invention encompasses the oligonucleotides disclosed in Table 4 and Table 5 as well as variants of these oligonucleotides s with 5′-end variations.

TABLE 9
Oligonucleotides for VHR HLA typing

| | SEQ | | |
|---|---|---|---|
| | ID | | |
| HLA target | NO: | Primer Name | HLA-hybridizing Sequence |
| HLA-A Exon 1 5′ | 129 | PM1283 | GTTTCCAGAGAAGCCAATCAGTGTCGT |
| HLA-A Exon 1 3′ | 130 | RHLAA1TV3 | GAGCCTGGGGICGAGSAGT* |
| HLA-B Exon 1 5′ | 131 | FHLAB1-2TV2 | CCACCCGGACTCAGARTCTCCT* |
| HLA-B Exon 1 3′ | 132 | RHLAB1TV1 | GGTGTAGAAATACCTCATGGAGTGGGAGC |
| HLA-C Exon 1 5′ | 133 | FHLAC1-2TV3 | TTCTAGAGAAGCCAATCAGCGTCT |
| HLA-C Exon 1 3′ | 134 | RHLAC1TV1 | ACTGCGTGTCGTCCACGTAGCCCA |
| HLA-B Exon 5 5′ | 135 | FHLAB5TV1 | GGGTAAGGAGGGGGATGAGGGGTCATA |
| HLA-B Exon 5 3′ | 136 | RHLAB4-5TV1 | GCTGCTTCCCAGTAATGAGGCAGGGA |
| HLA-C Exon 5 5′ | 137 | FHLAC5TV3 | GAAGAGCAGAGATACACGTGCCAT |
| HLA-C Exon 5 3′ | 138 | RHLAC4-5TV5 | CACTTCTACCTGGGGCTTGAAACT |
| HLA-C Exon 6-7 5′ | 139 | FHLAC6-7TV2 | TGGGTCCAAGACTAGGAGGTTCCC |
| DPA1 Exon 2 5′ | 140 | PM1273 | GACCACTTGCATATTCAAACTGACA |
| DPA1 Exon 2 3′ | 141 | PM059 | GGCTACAGAGGAAGAGGCAAAGATAGG |
| DRB1 Exon 3 5′ | 142 | PM1332 | GCCTRGGTTTRCAGAGAAGCAGACA* |
| DRB1 Exon 3 3′ | 143 | PM1312 | AGAAATTTAGGAAGTCAGAAAGCTGCTCA |
| DRB1 Exon 3 3′ | 144 | PMBNH1336 | GCCTAGGTTTACAGAGAAGCAGACA* |

*S = C or G. The primer sample contains a mixture of equal amounts of oligonucleotides containing C or G at the indicated position.
I = inosine
R = C or T. The primer sample contains a mixture of equal amounts of oligonucleotides containing C or T at the indicated position

In some embodiments, the oligonucleotides of the present invention are incorporated into fusion primers further comprising additional (non-HLA hybridizing) sequences. Such additional sequences are placed at the 5′-end of the HLA hybridizing sequences disclosed in Tables 4, 5 and 9. The additional (non-HLA hybridizing) sequences enable various uses of the oligonucleotides including, for example, Next Generation Sequencing methods (NGS), digital droplet PCR methods (ddPCR), “four primer” amplification methods or targeted resequencing methods. The additional sequences include without limitation, adaptor sequences, library key tag sequences and multiplex identification tag (MID) sequences. Examples of adaptor and MID sequences are disclosed in Tables 3 and 10. Generally, such additional sequences are developed and made publicly available by developers of the instruments on which the method employing the sequences is to be practiced. The additional sequences may be obtained e.g., from 454 Life Sciences (Branford, Conn.), Life Technologies (Grand Island, N.Y.), Illumina (San Diego, Calif.), Bio-Rad Labs. (Hercules, Calif.), or Fluidigm (South San Francisco, Calif.)

TABLE 10
Non-HLA sequences in primers

| | SEQ | |
|---|---|---|
| | ID | |
| Function | NO: | SEQUENCE |
| Adaptor | 145 | GCCTCCCTCGCGCCA |
| Adaptor | 146 | ACACTGACGACATGGTTCTACA |
| Library Key Tag | 147 | TCAG |
| MID | 148 | ACGAGTGCGT |

In another embodiment, the invention is a set of fusion oligonucleotides specifically hybridizing to and capable of directing amplification of one or more genes and exons selected from HLA-A, Exon 1, 5, HLA-B, Exon 1, 5, HLA-C, Exons 1, 5, 6, 7, DRB1, Exon 3, DRB3, Exon 3, DRB4, Exon 3, DRB5, Exon 3, and DPA1, Exon 2, the set comprising one or more pairs of oligonucleotides having a non-HLA-hybridizing sequence and an HLA hybridizing sequence disclosed in Table 9. In some embodiments, the non-HLA-hybridizing sequence comprises one or more of adaptor, a library key and an MID. In variations of this embodiment, the invention is a set of fusion oligonucleotides specifically hybridizing to and capable of directing amplification of HLA-A, Exon 1, 5, HLA-B, Exon 1, 5, HLA-C, Exons 1, 5, 6, 7, DRB1, Exon 3, DRB3, Exon 3, DRB4, Exon 3, DRB5, Exon 3, and DPA1, Exon 2, the set comprising the fusion oligonucleotides comprising a non-HLA-hybridizing sequence and HLA-hybridizing sequences disclosed in Table 9. In other variations of this embodiment, the invention is a set of oligonucleotides specifically hybridizing to and capable of directing amplification of HLA-A, Exon 1, 5, HLA-B, Exon 1, 5, HLA-C, Exons 1, 5, 6, 7, DRB1, Exon 3, DRB3, Exon 3, DRB4, Exon 3, DRB5, Exon 3, and DPA1, Exon 2, the set consisting of the fusion oligonucleotides comprising a non-HLA-hybridizing sequence and HLA-hybridizing sequences disclosed in Table 9.

The set of oligonucleotides of the present invention may be a part of a larger set of fusion oligonucleotides, i.e., the set disclosed herein may be combined with additional oligonucleotides specifically hybridizing to and directing amplification of other HLA sequences. In some embodiments, the invention is a set of oligonucleotides specifically hybridizing to and capable of directing amplification of one or more genes and exons selected from HLA-A, Exons 1, 2, 3, 4, 5, HLA-B, Exons 1, 2, 3, 4, 5, HLA-C, Exons 1, 2, 3, 4, 5, 6, 7, DQB1, Exon 2, 3, DRB1, Exons 2, 3, DRB3, Exons 2, 3, DRB4, Exons 2, 3, DRB5, Exons 2, 3, DPA1, Exon 2, and DPB1, Exon 2, the set being a combination one or more pairs of fusion oligonucleotides having a non-HLA-hybridizing sequence and an HLA hybridizing sequence disclosed in Table 9 and one or more pairs of fusion oligonucleotides having a non-HLA-hybridizing sequence and an HLA hybridizing sequence disclosed in Table 4 or Table 5. In variations of this embodiment, the invention is a set of fusion oligonucleotides specifically hybridizing to and capable of directing amplification of HLA-A, Exons 1, 2, 3, 4, 5, HLA-B, Exons 1, 2, 3, 4, 5, HLA-C, Exons 1, 2, 3, 4, 5, 6, 7, DQB1, Exon 2, 3, DRB1, Exons 2, 3, DRB3, Exons 2, 3, DRB4, Exons 2, 3, DRB5, Exons 2, 3, DPA1, Exon 2, and DPB1, Exon 2, the set comprising a combination of fusion oligonucleotides having a non-HLA-hybridizing sequence and an HLA hybridizing sequence disclosed in Table 9 and fusion oligonucleotides having a non-HLA-hybridizing sequence and an HLA hybridizing sequence disclosed in Table 4 or Table 5. In further variations of this embodiment, the invention is a set of oligonucleotides specifically hybridizing to and capable of directing amplification of HLA-A, Exons 1, 2, 3, 4, 5, HLA-B, Exons 1, 2, 3, 4, 5, HLA-C, Exons 1, 2, 3, 4, 5, 6, 7, DQB1, Exon 2, 3, DRB1, Exons 2, 3, DRB3, Exons 2, 3, DRB4, Exons 2, 3, DRB5, Exons 2, 3, DPA1, Exon 2, and DPB1, Exon 2, the set consisting of a combination of fusion oligonucleotides having a non-HLA-hybridizing sequence and an HLA hybridizing sequence disclosed in Table 9 and fusion oligonucleotides having a non-HLA-hybridizing sequence and an HLA hybridizing sequence disclosed in Table 4. In further variations of this embodiment, the invention is a set of oligonucleotides specifically hybridizing to and capable of directing amplification of HLA-A, Exons 1, 2, 3, 4, 5, HLA-B, Exons 1, 2, 3, 4, 5, HLA-C, Exons 1, 2, 3, 4, 5, 6, 7, DQB1, Exon 2, 3, DRB1, Exons 2, 3, DRB3, Exons 2, 3, DRB4, Exons 2, 3, DRB5, Exons 2, 3, DPA1, Exon 2, and DPB1, Exon 2, the set consisting of a combination of fusion oligonucleotides having a non-HLA-hybridizing sequence and an HLA hybridizing sequence disclosed in Table 9 and fusion oligonucleotides having a non-HLA-hybridizing sequence and an HLA hybridizing sequence disclosed in Table 5.

Kits

In yet another embodiment, the invention is a kit containing reagents necessary for very high resolution (VHR) HLA genotyping. The kit includes a set of fusion oligonucleotides for determining an individual's HLA genotype at HLA-A, Exon 1, 5, HLA-B, Exon 1, 5, HLA-C, Exons 1, 5, 6, 7, DRB1, Exon 3, DRB3, Exon 3, DRB4, Exon 3, DRB5, Exon 3, and DPA1, Exon 2, comprising one or more pairs of oligonucleotides, each having a non-HLA-hybridizing sequence and an HLA hybridizing sequence disclosed in Table 9. In some embodiments, the non-HLA-hybridizing sequence comprises one or more of adaptor, a library key and an MID. In variations of this embodiment, the kit includes a set of fusion oligonucleotides consisting of oligonucleotides having the HLA-hybridizing sequences disclosed in Table 9.

The kit of the present invention may contain a larger set of fusion oligonucleotides. In some embodiments, the invention the kit includes a set of fusion oligonucleotides for determining an individual's HLA genotype at HLA-A, Exons 1, 2, 3, 4, 5, HLA-B, Exons 1, 2, 3, 4, 5, HLA-C, Exons 1, 2, 3, 4, 5, 6, 7, DQB1, Exon 2, 3, DRB1, Exons 2, 3, DRB3, Exons 2, 3, DRB4, Exons 2, 3, DRB5, Exons 2, 3, DPA1, Exon 2, and DPB1, Exon 2, each oligonucleotide having a non-HLA-hybridizing sequence and an HLA hybridizing sequence disclosed in Table 9 and one or more pairs of fusion oligonucleotides having a non-HLA-hybridizing sequence and an HLA hybridizing sequence disclosed in Table 4 or Table 5. In variations of this embodiment, the kit includes a set of fusion oligonucleotides comprising oligonucleotides having the HLA hybridizing sequence disclosed in Table 9 and oligonucleotides having the HLA hybridizing sequence disclosed in Table 4 or Table 5. In further variations of this embodiment, the kit includes a set of fusion oligonucleotides consisting of oligonucleotides having the HLA hybridizing sequence disclosed in Table 9 and oligonucleotides having the HLA hybridizing sequence disclosed in Table 4. In further variations of this embodiment, the kit includes a set of fusion oligonucleotides consisting of oligonucleotides having the HLA hybridizing sequence disclosed in Table 9 and oligonucleotides having the HLA hybridizing sequence disclosed in Table 5.

The kit further comprises one or more reagents necessary for the performance of amplification and detection assay, such as nucleoside triphosphates, nucleic acid polymerase and buffers necessary for the function of the polymerase.

### Methods of HLA Genotyping

In another embodiment, the invention is a method of determining HLA genotype of an individual with very high resolution (VHR) using oligonucleotides of the present invention. The method comprises amplifying the HLA sequences in the sample derived from the individual using the oligonucleotides in Tables 4 or Table 5 and 9 and determining the sequences of the HLA amplicons thereby determining the individual's HLA genotype with very high resolution.

In some embodiments, the sequence of the HLA amplicons is determined by clonal sequencing methods including Next Generation Sequencing (NGS) methods also known as Multiplex Parallel Sequencing (MPS) methods. In some embodiments, the invention is a method of determining HLA genotype of an individual with very high resolution, the method comprising amplifying the HLA sequences in the sample derived from the individual using fusion oligonucleotides comprising the HLA-hybridizing sequences disclosed in Tables 4, 5 and 9 and using any of clonal analysis technique, e.g., the sequencing technique described above, or clonal amplification comprising a two-dimensional surface-based (e.g., slide-based) amplification as described e.g., in U.S. Pat. Nos. 7,835,871, 8,244,479, 8,315,817 and 8,412,467; or any other clonal sequence analysis method that is or will become available to determine the sequences of the HLA amplicons thereby determining the individual's HLA genotype with very high resolution. In some embodiments, the amplicons are sequenced by one of the methods selected from a base-incorporation method, e.g., a pyrosequencing method (U.S. Pat. Nos. 6,274,320, 6,258,568 and 6,210,891); a hydrogen ion detection method (ISFET) (e.g., U.S. Pat. No. 8,262,900), or a dye-terminator detection method (U.S. Pat. Nos. 7,835,871, 8,244,479, 8,315,817 and 8,412,467.) Steps (b)-(d) or equivalents thereof may be performed using any available deep sequencing technology and instrument (i.e., technology and instrument capable of digital sequence readout). Without limitation, the examples of instruments include GS family of instruments (454 Life Sciences, Branford, Conn.); ION PROTON® and PGM™ (Life Technologies, Grand Island, N.Y.); HISEQ® and MISEQ® (Illumina, San Diego, Calif.) or any improvements and modifications of thereof.

### EXAMPLES

Without limiting the invention to a single technology or instrument but merely by way of example, an embodiment of the method may be performed using the GS family of sequencing instruments including GS FLX®, GS FLX+®, GS FLX TITANIUM® or GS Junior® (454 Life Sciences, Branford, Conn.) as described below.

### Example 1

### Identification of HLA Genotypes Using Massive Parallel Sequencing

DNA Isolation

DNA was purified from cell lines using the GENTRA° PUREGENF kit (Qiagen, Valencia, Calif.).

Genomic PCR

PCR amplifications were carried out in individual 25 μl reactions with 1-20 ng of DNA template and 10 pmoles each of forward/reverse “fusion” primers (i.e., primers containing HLA-hybridizing portion and adaptor portion as described herein), 10 mM Tris-HCl, pH 8.3, 50 mM KCl, 1.5 mM MgCl, 600 μM dNTPs (150 μM each of dA, dC, dG and dUTP), glycerol 10% w/v, AMPLITAQ® Gold (2 units) DNA polymerase. Thermal cycling conditions were: 95° C.-10 min; 31 cycles of 95° C.-15 sec, 60° C.-45 sec, 72° C.-15 sec; 72° C.-5 min in an ABI GENEAMP® PCR System 9700 (Life Technologies, Carlsbad, Calif.). Molecular biology grade water was used to make all solutions.

Amplicon Cleanup, Quantification, Dilution and Pooling

Short, non-specific and primer-dimer artifact products were removed from the amplicons using the AMPURE® system (Agencourt Bioscience Corp., Beverly, Mass.), following the protocol for cleanup described in the 454/Roche Applied Science GS GType HLA MR and HR kits. (Roche Applied Science, Indianapolis, Ind.). Aliquots of purified amplicons were further evaluated by electrophoresis on a 96 well E-Gel® (Life Technologies, Carlsbad, Calif.). If primer dimers were observed the AMPure step was repeated and product reevaluated by E-gel.

The purified amplicons were then quantified by QUANT-IT™ PICOGREEN® assay (Life Technologies, Carlsbad, Calif.) on a microplate spectrofluorimeter. Eight standards spanned DNA concentrations from 0 to 12.5 ng/μl. Any amplicons that could not be detected by PICOGREEN® were assigned a concentration of 0.1 ng/μl (in order to allow a dilution calculation to be made) and carried through subsequent steps. Amplicons were diluted to 1×10⁶ molecules/μl. Pools of amplicons were made such that all amplicons destined for sequencing on a single region of the 454 PicoTiter Plate (PTP) were pooled; in general 5 μl of each amplicon was added to a pool.

Emulsion PCR, Bead Recovery and Pyrosequencing

Emulsion PCR (emPCR), enrichment of DNA containing beads, and pyrosequencing on the GS FLX instrument were carried out on a 4 region PTP as per the 454 Life Sciences GS FLX Titanium Series manuals (Roche Applied Science, Indianapolis, Ind.): emPCR Method Manual—LibA MV (January 2010); Sequencing Method Manual (May 2010), with the following exceptions: 1) during emPCR, amplicon pools were used at 0.4-0.5 copies per bead, 2) the emPCR primer was used at a concentration of 0.25 times that specified, 3) bead enrichment was automated by use of the REMe module (Roche Applied Science, Indianapolis, Ind.) on a MultiProbe HT liquid handler (Perkin Elmer, Waltham, Mass.), and 4) for sequencing, 60% of the recommended load of enriched DNA beads was dispensed onto the PTP plate. Sequencing on the GS Junior was carried out in accordance with manufacturer's recommendations in GS Junior emPCR Amplification Method Manual Lib-A (March 2012) and GS Junior Sequencing Manual (January 2013).

Sequence Analysis

Sequences were consolidated using the consensus functions of 454 AVA software (Roche Applied Science, Indianapolis, Ind.). Conexio Genomics Assign software MPS and/or Assign ATF 454 v34 software (Conexio Genomics, Perth, Australia) installed on a Microsoft Windows® based computer, were used for analysis of sequences.

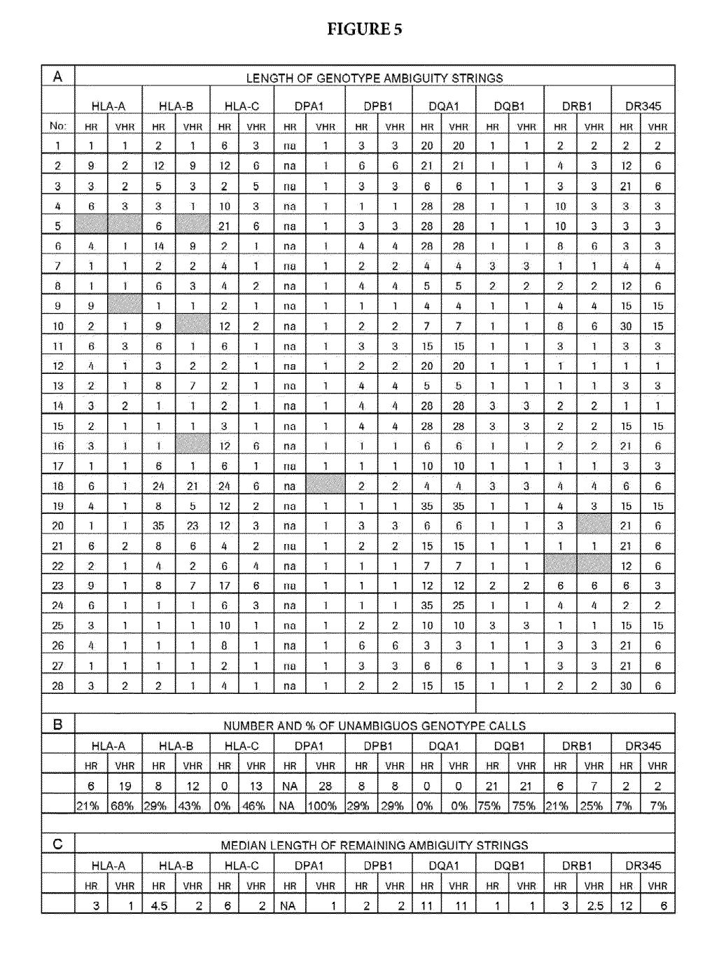

Results are shown in FIG. 5. Section A: For the sample Nos. 1-28, numerical entries indicate the length of the genotype ambiguity strings (lists of the possible genotypes for a given sample/locus in light of the data obtained) within the genes listed in the column headers when using High Resolution (HR) genotyping, the Very High Resolution (VHR) genotyping. For example, the increase in resolution, i.e., decrease in length in the ambiguity string, when using the VHR system can be seen for sample 7, gene HLA-C, where the genotype ambiguity string length is 4 for the HR system and is reduced to 1 for the VHR systems.

Section B: Numerical entries indicate the number and percentage of unambiguous genotype calls in the dataset shown in (A). For example, for HLA-A, the number of samples with unambiguous genotype calls was 6 (21% of samples) for HR typing, but increased to 19 (68% of samples) for VHR typing.

Section C. Numerical values indicate the median length of the genotype ambiguity string for those sample/loci combinations in the dataset that do not give unambiguous calls. For example, for HLA-A, the length of the ambiguity string was 3 with HR typing, but reduced to 1 with VHR typing.

While the invention has been described in detail with reference to specific examples, it will be apparent to one skilled in the art that various modifications can be made within the scope of this invention. Thus the scope of the invention should not be limited by the examples described herein, but by the claims presented below.

We claim:

1. A method of determining HLA genotype of at least one individual for HLA-A, HLA-B, HLA-C, DQB1, DRB1, DRB3, DRB4, DRB5, DPA1 and DPB1 the method comprising:
a. amplifying sequences of HLA-A, Exons 1, 2, 3, 4, 5, HLA-B, Exons 1, 2, 3, 4, 5, HLA-C, Exons 1, 2, 3, 4, 5, 6, 7, DQB1, Exons 2, 3, DRB1, Exons 2, 3, DRB3, Exons 2, 3, DRB4, Exons 2, 3, DRB5, Exons 2, 3, DPA1, Exon 2, and DPB1, Exon 2 using oligonucleotides comprising the HLA hybridizing sequences of SEQ ID NOs: 23-144 to generate HLA amplicons
b. determining the sequence of the HLA amplicons amplified in step a.
c. assigning the HLA alleles to the individual thereby determining the HLA genotype of the individual.

2. The method of claim 1, wherein the oligonucleotides comprising the HLA hybridizing sequences further comprise an adapter sequence, and a molecular identification sequence (MID).

3. The method of claim 2, wherein the adapter region is selected from adaptor sequences listed in Tables 3 and 10.

4. The method of claim 2, wherein the molecular identification sequence (MID) is selected from MID sequences listed in Table 10.

5. A kit comprising primer pairs for obtaining HLA amplicons for determining HLA genotype of at least one individual for HLA-A, HLA-B, HLA-C, DRB1, DRB3, DRB4, DRB5 and DPA1 comprising fusion oligonucleotides having the HLA hybridizing region of SEQ ID NOs: 129-144.

6. The kit of claim 5 comprising the primer pairs consisting of fusion oligonucleotides having the HLA hybridizing region of SEQ ID NO: 129-144.

7. A kit comprising primer pairs for obtaining HLA amplicons for determining HLA genotype of at least one individual for HLA-A, HLA-B, HLA-C, DQB1, DRB1, DRB3, DRB4, DRB5, DPA1 and DPB1 comprising fusion oligonucleotides having the HLA hybridizing region of SEQ ID NOs: 23-144.

8. The kit of claim 7 comprising the primer pairs consisting of fusion oligonucleotides having the HLA hybridizing region of SEQ ID NOs: 23-144.

9. The kit of claim 7 wherein the oligonucleotides further comprise an adapter sequence, and a molecular identification sequence (MID).

10. The kit of claim 9, wherein the adapter region is selected from the adaptor sequences listed in Tables 3 and 10.

11. The kit of claim 9, wherein the molecular identification sequence (MID) is selected from MID sequences listed in Table 10.

12. A method of obtaining HLA amplicons for determining HLA genotype of at least one individual at HLA-A, Exons 1, 2, 3, 4, 5, HLA-B, Exons 1, 2, 3, 4, 5, HLA-C, Exons 1, 2, 3, 4, 5, 6, 7, DQB1, Exons 2, 3, DRB1, Exons 2, 3, DRB3, Exons 2, 3, DRB4, Exons 2, 3, DRB5, Exons 2, 3, DPA1, Exon 2, and DPB1, Exon 2 comprising the steps of:
a. amplifying a plurality of first amplicons from a sample derived from the individual, wherein the first amplicons are amplified with a plurality of pairs of nucleic acid primers selected from SEQ ID NOs: 23-144;
b. amplifying the first amplicons to produce a plurality of populations of second amplicons, wherein each population of second amplicons is clonally amplified from one of the first amplicons;
c. sequencing the plurality of populations of second amplicons to generate a nucleic acid sequence composition for each of the plurality of second amplicons; and
d. assigning the HLA alleles to the individual thereby determining the HLA genotype of the individual.

13. The method of claim 12, wherein said plurality of pairs of nucleic acid primers comprises SEQ ID NOs: 23-68 and 129-144.

14. The method of claim 12, wherein said plurality of pairs of nucleic acid primers consists of SEQ ID NOs: 69-144.

15. A set of oligonucleotides for obtaining HLA amplicons at HLA-A, HLA-B, HLA-C, DRB1, DRB3, DRB4, DRB5 and DPA1 comprising one or more pairs of fusion oligonucleotides having the HLA hybridizing region of SEQ ID NO: 129-144.

16. The set of claim 15 comprising oligonucleotides having the HLA hybridizing region of SEQ ID NO: 129-144.

17. The set of claim 15 consisting of oligonucleotides having the HLA hybridizing region of SEQ ID NO: Listed in Table 9.

18. A set of oligonucleotides for obtaining HLA amplicons at HLA-A, HLA-B, HLA-C, DQB1, DRB1, DRB3, DRB4, DRB5, DPA1 and DPB1 comprising one or more pairs of oligonucleotides having the HLA hybridizing region of SEQ ID NO: 23-144.

19. The set of claim 18, consisting of oligonucleotides having the HLA hybridizing region of SEQ ID NO: 23-68 and 129-144.

20. The set of claim 18 consisting of oligonucleotides having the HLA hybridizing region of SEQ ID NO: 69-144.

21. The set of claim 18 wherein the oligonucleotides further comprise an adapter sequence, and a molecular identification sequence (MID).

22. The set of claim 21, wherein the adapter region is selected from the adaptor sequences listed in Tables 3 and 10.

23. The set of claim 21, wherein the molecular identification sequence (MID) is selected from MID sequences listed in Table 10.


SEQUENCE LISTING (148 sequences)

SEQ ID NO: 1 - DNA, 25 bp, Artificial Sequence
  Description of Artificial Sequence Synthetic oligonucleotide
gcctccctcgcgccatccgactcag

SEQ ID NO: 2 - DNA, 25 bp, Artificial Sequence
  Description of Artificial Sequence Synthetic oligonucleotide
gccttgccagcccgcgcagtctcag

SEQ ID NO: 3 - DNA, 25 bp, Artificial Sequence
  Description of Artificial Sequence Synthetic oligonucleotide
cgtatcgcctccctcgcgccatcag

SEQ ID NO: 4 - DNA, 25 bp, Artificial Sequence
  Description of Artificial Sequence Synthetic oligonucleotide
ctatgcgccttgccagcccgctcag

SEQ ID NO: 5 - DNA, 10 bp, Artificial Sequence
  Description of Artificial Sequence Synthetic oligonucleotide
acgctcgaca

SEQ ID NO: 6 - DNA, 10 bp, Artificial Sequence
  Description of Artificial Sequence Synthetic oligonucleotide
agacgcactc

SEQ ID NO: 7 - DNA, 10 bp, Artificial Sequence
  Description of Artificial Sequence Synthetic oligonucleotide
agcactgtag

SEQ ID NO: 8 - DNA, 10 bp, Artificial Sequence
  Description of Artificial Sequence Synthetic oligonucleotide
atcagacacg

SEQ ID NO: 9 - DNA, 10 bp, Artificial Sequence
  Description of Artificial Sequence Synthetic oligonucleotide
atatcgcgag

SEQ ID NO: 10 - DNA, 10 bp, Artificial Sequence
  Description of Artificial Sequence Synthetic oligonucleotide
cgtgtctcta

SEQ ID NO: 11 - DNA, 10 bp, Artificial Sequence
  Description of Artificial Sequence Synthetic oligonucleotide
ctcgcgtgtc

SEQ ID NO: 12 - DNA, 10 bp, Artificial Sequence
  Description of Artificial Sequence Synthetic oligonucleotide
tagtatcagc

SEQ ID NO: 13 - DNA, 10 bp, Artificial Sequence
  Description of Artificial Sequence Synthetic oligonucleotide
tctctatgcg

SEQ ID NO: 14 - DNA, 10 bp, Artificial Sequence
  Description of Artificial Sequence Synthetic oligonucleotide
tactgagcta

SEQ ID NO: 15 - DNA, 10 bp, Artificial Sequence
  Description of Artificial Sequence Synthetic oligonucleotide
catagtagtg

SEQ ID NO: 16 - DNA, 10 bp, Artificial Sequence
  Description of Artificial Sequence Synthetic oligonucleotide
cgagagatac

SEQ ID NO: 17 - DNA, 10 bp, Artificial Sequence
  Description of Artificial Sequence Synthetic oligonucleotide
acgagtgcgt

SEQ ID NO: 18 - DNA, 10 bp, Artificial Sequence
  Description of Artificial Sequence Synthetic oligonucleotide
tgatacgtct

SEQ ID NO: 19 - DNA, 10 bp, Artificial Sequence
  Description of Artificial Sequence Synthetic oligonucleotide
cgtgtctctg

SEQ ID NO: 20 - DNA, 10 bp, Artificial Sequence
  Description of Artificial Sequence Synthetic oligonucleotide
catagtagta

SEQ ID NO: 21 - DNA, 10 bp, Artificial Sequence
  Description of Artificial Sequence Synthetic oligonucleotide
acgagtgcga

SEQ ID NO: 22 - DNA, 10 bp, Artificial Sequence
  Description of Artificial Sequence Synthetic oligonucleotide
catagtagtc

SEQ ID NO: 23 - DNA, 26 bp, Artificial Sequence
  Description of Artificial Sequence Synthetic primer
gaaacggcctctgtggggagaagcaa

SEQ ID NO: 24 - DNA, 25 bp, Artificial Sequence
  Description of Artificial Sequence Synthetic primer
ggtggatctcggacccggagactgt

SEQ ID NO: 25 - DNA, 20 bp, Artificial Sequence
  Description of Artificial Sequence Synthetic primer
gactgggctgaccgtggggt

SEQ ID NO: 26 - DNA, 22 bp, Artificial Sequence
  Description of Artificial Sequence Synthetic primer
cccctggtaccvgtgcgctgca

SEQ ID NO: 27 - DNA, 20 bp, Artificial Sequence
  Description of Artificial Sequence Synthetic primer
gactgggctgacckyggggt

SEQ ID NO: 28 - DNA, 28 bp, Artificial Sequence
  Description of Artificial Sequence Synthetic primer
gagggtgatattctagtgttggtcccaa

SEQ ID NO: 29 - DNA, 30 bp, Artificial Sequence
  Description of Artificial Sequence Synthetic primer
tgcctgaatgwtctgactcttcccgtmaga

SEQ ID NO: 30 - DNA, 24 bp, Artificial Sequence
  Description of Artificial Sequence Synthetic primer
tgaccctgctaaaggtctccagag

SEQ ID NO: 31 - DNA, 25 bp, Artificial Sequence
  Description of Artificial Sequence Synthetic primer
ctgggttctgtgctcycttccccat

SEQ ID NO: 32 - DNA, 26 bp, Artificial Sequence
  Description of Artificial Sequence Synthetic primer
ctccagagaggctcctgctttccsta

SEQ ID NO: 33 - DNA, 29 bp, Artificial Sequence
  Description of Artificial Sequence Synthetic primer
agagctcgggaggagcgaggggaccscag

SEQ ID NO: 34 - DNA, 27 bp, Artificial Sequence
  Description of Artificial Sequence Synthetic primer
actcgaggcctcgctctggttgtagta

SEQ ID NO: 35 - DNA, 17 bp, Artificial Sequence
  Description of Artificial Sequence Synthetic primer
cggtcgagggtytgggc

SEQ ID NO: 36 - DNA, 23 bp, Artificial Sequence
  Description of Artificial Sequence Synthetic primer
agagctcgggccagggtctcaca

SEQ ID NO: 37 - DNA, 30 bp, Artificial Sequence
  Description of Artificial Sequence Synthetic primer
actcgagggaggccatccccggcgacctat

SEQ ID NO: 38 - DNA, 23 bp, Artificial Sequence
  Description of Artificial Sequence Synthetic primer
cccggtttcattttcagttgagg

SEQ ID NO: 39 - DNA, 26 bp, Artificial Sequence
  Description of Artificial Sequence Synthetic primer
gcgcctgaattttctgactcttccca

SEQ ID NO: 40 - DNA, 21 bp, Artificial Sequence
  Description of Artificial Sequence Synthetic primer
ggctcctgctttccctgagaa

SEQ ID NO: 41 - DNA, 19 bp, Artificial Sequence
  Description of Artificial Sequence Synthetic primer
ctggtcacatgggtggtcc

SEQ ID NO: 42 - DNA, 19 bp, Artificial Sequence
  Description of Artificial Sequence Synthetic primer
agatatgacccctcatccc

SEQ ID NO: 43 - DNA, 24 bp, Artificial Sequence
  Description of Artificial Sequence Synthetic primer
agtcgacgaadcggcctctgsgga

SEQ ID NO: 44 - DNA, 24 bp, Artificial Sequence
  Description of Artificial Sequence Synthetic primer
actcgaggggcyggggtcactcac

SEQ ID NO: 45 - DNA, 24 bp, Artificial Sequence
  Description of Artificial Sequence Synthetic primer
acgtcgacgggccaggktctcaca

SEQ ID NO: 46 - DNA, 26 bp, Artificial Sequence
  Description of Artificial Sequence Synthetic primer
acctcgaggtcagcagcctgaccaca

SEQ ID NO: 47 - DNA, 20 bp, Artificial Sequence
  Description of Artificial Sequence Synthetic primer
ctccccactgcccctggtac

SEQ ID NO: 48 - DNA, 29 bp, Artificial Sequence
  Description of Artificial Sequence Synthetic primer
caaagtgtctgaattttctgactcttccc

SEQ ID NO: 49 - DNA, 21 bp, Artificial Sequence
  Description of Artificial Sequence Synthetic primer
tgaagggctccagaaggactt

SEQ ID NO: 50 - DNA, 18 bp, Artificial Sequence
  Description of Artificial Sequence Synthetic primer
tgaagggctccaggactt

SEQ ID NO: 51 - DNA, 25 bp, Artificial Sequence
  Description of Artificial Sequence Synthetic primer
gtgtcgcaagagagatrcaaagtgt

SEQ ID NO: 52 - DNA, 19 bp, Artificial Sequence
  Description of Artificial Sequence Synthetic primer
gaggrgaaggtgaggggcc

SEQ ID NO: 53 - DNA, 28 bp, Artificial Sequence
  Description of Artificial Sequence Synthetic primer
gctgcaggagagtggcgcctccgctcat

SEQ ID NO: 54 - DNA, 25 bp, Artificial Sequence
  Description of Artificial Sequence Synthetic primer
cggatccggcccaaagccctcactc

SEQ ID NO: 55 - DNA, 29 bp, Artificial Sequence
  Description of Artificial Sequence Synthetic primer
gtttcttycatcattttgtgtattaaggt

SEQ ID NO: 56 - DNA, 20 bp, Artificial Sequence
  Description of Artificial Sequence Synthetic primer
cggtagagttgtagcgttta

SEQ ID NO: 57 - DNA, 33 bp, Artificial Sequence
  Description of Artificial Sequence Synthetic primer
gtcagtttcttycatcattttgtgtattaaggt

SEQ ID NO: 58 - DNA, 34 bp, Artificial Sequence
  Description of Artificial Sequence Synthetic primer
gaaagtcagtttcttycatcattttgtgtattaa

SEQ ID NO: 59 - DNA, 24 bp, Artificial Sequence
  Description of Artificial Sequence Synthetic primer
ccatgasaagatctggggacctct

SEQ ID NO: 60 - DNA, 29 bp, Artificial Sequence
  Description of Artificial Sequence Synthetic primer
aggatccccgcagaggatttcgtgtacca

SEQ ID NO: 61 - DNA, 29 bp, Artificial Sequence
  Description of Artificial Sequence Synthetic primer
tcctgcaggacgctcacctctccgctgca

SEQ ID NO: 62 - DNA, 23 bp, Artificial Sequence
  Description of Artificial Sequence Synthetic primer
tggagcccacagtgaccatctcc

SEQ ID NO: 63 - DNA, 21 bp, Artificial Sequence
  Description of Artificial Sequence Synthetic primer
gctggggtgctccacgtggca

SEQ ID NO: 64 - DNA, 27 bp, Artificial Sequence
  Description of Artificial Sequence Synthetic primer
agtgacatcagggataagagatgggaa

SEQ ID NO: 65 - DNA, 27 bp, Artificial Sequence
  Description of Artificial Sequence Synthetic primer
ccggatccttcgtgtccccacagcacg

SEQ ID NO: 66 - DNA, 28 bp, Artificial Sequence
  Description of Artificial Sequence Synthetic primer
ccgaattccgctgcactgtgaagctctc

SEQ ID NO: 67 - DNA, 23 bp, Artificial Sequence
  Description of Artificial Sequence Synthetic primer
ccggatccttcgtgtccccacag

SEQ ID NO: 68 - DNA, 24 bp, Artificial Sequence
  Description of Artificial Sequence Synthetic primer
gattctraatgctcacagatggcg

SEQ ID NO: 69 - DNA, 27 bp, Artificial Sequence
  Description of Artificial Sequence Synthetic primer
gtttccagagaagccaatcagtgtcgt

SEQ ID NO: 70 - DNA, 22 bp, Artificial Sequence
  Description of Artificial Sequence Synthetic primer
taaagtccgcacgcacccaccg

SEQ ID NO: 71 - DNA, 27 bp, Artificial Sequence
  Description of Artificial Sequence Synthetic primer
cttggaaccctcagtgagacaagaaat

SEQ ID NO: 72 - DNA, 26 bp, Artificial Sequence
  Description of Artificial Sequence Synthetic primer
ttggaaccctcagtgagacaagaaat

SEQ ID NO: 73 - DNA, 23 bp, Artificial Sequence
  Description of Artificial Sequence Synthetic primer
ctggggcttggaaccctcagtga

SEQ ID NO: 74 - DNA, 22 bp, Artificial Sequence
  Description of Artificial Sequence Synthetic primer
ggttctgtgctcycttccccat

SEQ ID NO: 75 - DNA, 24 bp, Artificial Sequence
  Description of Artificial Sequence Synthetic primer
ggaaccctcagtgagacaagaaat

SEQ ID NO: 76 - DNA, 20 bp, Artificial Sequence
  Description of Artificial Sequence Synthetic primer
gggcttggaaccctcagtga

SEQ ID NO: 77 - DNA, 26 bp, Artificial Sequence
  Description of Artificial Sequence Synthetic primer
gcacccacccggactcagartctcct

SEQ ID NO: 78 - DNA, 22 bp, Artificial Sequence
  Description of Artificial Sequence Synthetic primer
ccacccggactcagartctcct

SEQ ID NO: 79 - DNA, 19 bp, Artificial Sequence
  Description of Artificial Sequence Synthetic primer
ccgggccggggtcactcac

SEQ ID NO: 80 - DNA, 17 bp, Artificial Sequence
  Description of Artificial Sequence Synthetic primer
gggccggggtcactcac

SEQ ID NO: 81 - DNA, 20 bp, Artificial Sequence
  Description of Artificial Sequence Synthetic primer
cccgcggggattttggcctc

SEQ ID NO: 82 - DNA, 18 bp, Artificial Sequence
  Description of Artificial Sequence Synthetic primer
cgcggggattttggcctc

SEQ ID NO: 83 - DNA, 28 bp, Artificial Sequence
  Description of Artificial Sequence Synthetic primer
cgcgtttacccggtttcattttcagttg

SEQ ID NO: 84 - DNA, 26 bp, Artificial Sequence
  Description of Artificial Sequence Synthetic primer
cgtttacccggtttcattttcagttg

SEQ ID NO: 85 - DNA, 27 bp, Artificial Sequence
  Description of Artificial Sequence Synthetic primer
cccggtttcattttcagttgaggycaa

SEQ ID NO: 86 - DNA, 24 bp, Artificial Sequence
  Description of Artificial Sequence Synthetic primer
ggtttcattttcagttgaggycaa

SEQ ID NO: 87 - DNA, 23 bp, Artificial Sequence
  Description of Artificial Sequence Synthetic primer
ggagatggggaaggctccccact

SEQ ID NO: 88 - DNA, 19 bp, Artificial Sequence
  Description of Artificial Sequence Synthetic primer
atggggaaggctccccact

SEQ ID NO: 89 - DNA, 21 bp, Artificial Sequence
  Description of Artificial Sequence Synthetic primer
agggggccctcagaggaaact

SEQ ID NO: 90 - DNA, 22 bp, Artificial Sequence
  Description of Artificial Sequence Synthetic primer
gtttaggccaaaatccccgcgg

SEQ ID NO: 91 - DNA, 29 bp, Artificial Sequence
  Description of Artificial Sequence Synthetic primer
aaagcgcctgaattttctgactcttccca

SEQ ID NO: 92 - DNA, 25 bp, Artificial Sequence
  Description of Artificial Sequence Synthetic primer
cgcctgaattttctgactcttccca

SEQ ID NO: 93 - DNA, 26 bp, Artificial Sequence
  Description of Artificial Sequence Synthetic primer
gctgcttcccagtaatgaggcaggga

SEQ ID NO: 94 - DNA, 23 bp, Artificial Sequence
  Description of Artificial Sequence Synthetic primer
gcttcccagtaatgaggcaggga

SEQ ID NO: 95 - DNA, 23 bp, Artificial Sequence
  Description of Artificial Sequence Synthetic primer
tgcgttagcccctgtgtgsatgc

SEQ ID NO: 96 - DNA, 21 bp, Artificial Sequence
  Description of Artificial Sequence Synthetic primer
cgttagcccctgtgtgsatgc

SEQ ID NO: 97 - DNA, 28 bp, Artificial Sequence
  Description of Artificial Sequence Synthetic primer
cgggttctagagaagccaatcagcgtct

SEQ ID NO: 98 - DNA, 26 bp, Artificial Sequence
  Description of Artificial Sequence Synthetic primer
ggttctagagaagccaatcagcgtct

SEQ ID NO: 99 - DNA, 24 bp, Artificial Sequence
  Description of Artificial Sequence Synthetic primer
ttctagagaagccaatcagcgtct

SEQ ID NO: 100 - DNA, 21 bp, Artificial Sequence
  Description of Artificial Sequence Synthetic primer
ggtcgagggtctgggcgggtt

SEQ ID NO: 101 - DNA, 18 bp, Artificial Sequence
  Description of Artificial Sequence Synthetic primer
cgagggtctgggcgggtt

SEQ ID NO: 102 - DNA, 19 bp, Artificial Sequence
  Description of Artificial Sequence Synthetic primer
ccgggcyggggtcactcac

SEQ ID NO: 103 - DNA, 20 bp, Artificial Sequence
  Description of Artificial Sequence Synthetic primer
cgcccagaccctcgaccgga

SEQ ID NO: 104 - DNA, 18 bp, Artificial Sequence
  Description of Artificial Sequence Synthetic primer
cccagaccctcgaccgga

SEQ ID NO: 105 - DNA, 27 bp, Artificial Sequence
  Description of Artificial Sequence Synthetic primer
gagagaaaggtcagcagcctgaccaca

SEQ ID NO: 106 - DNA, 22 bp, Artificial Sequence
  Description of Artificial Sequence Synthetic primer
aaaggtcagcagcctgaccaca

SEQ ID NO: 107 - DNA, 23 bp, Artificial Sequence
  Description of Artificial Sequence Synthetic primer
cctcgaccggagagagcccyagt

SEQ ID NO: 108 - DNA, 20 bp, Artificial Sequence
  Description of Artificial Sequence Synthetic primer
cgaccggagagagcccyagt

SEQ ID NO: 109 - DNA, 26 bp, Artificial Sequence
  Description of Artificial Sequence Synthetic primer
tccattctcaggatggtcacatgggc

SEQ ID NO: 110 - DNA, 23 bp, Artificial Sequence
  Description of Artificial Sequence Synthetic primer
attctcaggatggtcacatgggc

SEQ ID NO: 111 - DNA, 29 bp, Artificial Sequence
  Description of Artificial Sequence Synthetic primer
gggcacacttctacctggggcttgaaact

SEQ ID NO: 112 - DNA, 26 bp, Artificial Sequence
  Description of Artificial Sequence Synthetic primer
cacacttctacctggggcttgaaact

SEQ ID NO: 113 - DNA, 22 bp, Artificial Sequence
  Description of Artificial Sequence Synthetic primer
cacacagggtcccaggctggga

SEQ ID NO: 114 - DNA, 19 bp, Artificial Sequence
  Description of Artificial Sequence Synthetic primer
acagggtcccaggctggga

SEQ ID NO: 115 - DNA, 32 bp, Artificial Sequence
  Description of Artificial Sequence Synthetic primer
acttctcttgggtccaagactaggaggttccc

SEQ ID NO: 116 - DNA, 24 bp, Artificial Sequence
  Description of Artificial Sequence Synthetic primer
tgggtccaagactaggaggttccc

SEQ ID NO: 117 - DNA, 22 bp, Artificial Sequence
  Description of Artificial Sequence Synthetic primer
cccacccccgaccacttcagct

SEQ ID NO: 118 - DNA, 20 bp, Artificial Sequence
  Description of Artificial Sequence Synthetic primer
cacccccgaccacttcagct

SEQ ID NO: 119 - DNA, 29 bp, Artificial Sequence
  Description of Artificial Sequence Synthetic primer
gaaacgtcccaatcaaagratccccatta

SEQ ID NO: 120 - DNA, 25 bp, Artificial Sequence
  Description of Artificial Sequence Synthetic primer
cgtcccaatcaaagratccccatta

SEQ ID NO: 121 - DNA, 23 bp, Artificial Sequence
  Description of Artificial Sequence Synthetic primer
gaccacttgcatattcaaactga

SEQ ID NO: 122 - DNA, 25 bp, Artificial Sequence
  Description of Artificial Sequence Synthetic primer
ggctacagaggaagaggcaaagata

SEQ ID NO: 123 - DNA, 25 bp, Artificial Sequence
  Description of Artificial Sequence Synthetic primer
gaccacttgcatattcaaactgaca

SEQ ID NO: 124 - DNA, 27 bp, Artificial Sequence
  Description of Artificial Sequence Synthetic primer
ggctacagaggaagaggcaaagatagg

SEQ ID NO: 125 - DNA, 21 bp, Artificial Sequence
  Description of Artificial Sequence Synthetic primer
cggatgctttgtggacccgca

SEQ ID NO: 126 - DNA, 20 bp, Artificial Sequence
  Description of Artificial Sequence Synthetic primer
ggatgctttgtggacccgca

SEQ ID NO: 127 - DNA, 30 bp, Artificial Sequence
  Description of Artificial Sequence Synthetic primer
ggatagagaggattctgaatgctcacagat

SEQ ID NO: 128 - DNA, 29 bp, Artificial Sequence
  Description of Artificial Sequence Synthetic primer
ggatagagaggattctgaatgctcacaga

SEQ ID NO: 129 - DNA, 27 bp, Artificial Sequence
  Description of Artificial Sequence Synthetic primer
gtttccagagaagccaatcagtgtcgt

SEQ ID NO: 130 - DNA, 19 bp, Artificial Sequence
  Description of Artificial Sequence Synthetic primer
gagcctggggncgagsagt

SEQ ID NO: 131 - DNA, 22 bp, Artificial Sequence
  Description of Artificial Sequence Synthetic primer
ccacccggactcagartctcct

SEQ ID NO: 132 - DNA, 29 bp, Artificial Sequence
  Description of Artificial Sequence Synthetic primer
ggtgtagaaatacctcatggagtgggagc

SEQ ID NO: 133 - DNA, 24 bp, Artificial Sequence
  Description of Artificial Sequence Synthetic primer
ttctagagaagccaatcagcgtct

SEQ ID NO: 134 - DNA, 24 bp, Artificial Sequence
  Description of Artificial Sequence Synthetic primer
actgcgtgtcgtccacgtagccca

SEQ ID NO: 135 - DNA, 27 bp, Artificial Sequence
  Description of Artificial Sequence Synthetic primer
gggtaaggagggggatgaggggtcata

SEQ ID NO: 136 - DNA, 26 bp, Artificial Sequence
  Description of Artificial Sequence Synthetic primer
gctgcttcccagtaatgaggcaggga

SEQ ID NO: 137 - DNA, 24 bp, Artificial Sequence
  Description of Artificial Sequence Synthetic primer
gaagagcagagatacacgtgccat

SEQ ID NO: 138 - DNA, 24 bp, Artificial Sequence
  Description of Artificial Sequence Synthetic primer
cacttctacctggggcttgaaact

SEQ ID NO: 139 - DNA, 24 bp, Artificial Sequence
  Description of Artificial Sequence Synthetic primer
tgggtccaagactaggaggttccc

SEQ ID NO: 140 - DNA, 25 bp, Artificial Sequence
  Description of Artificial Sequence Synthetic primer
gaccacttgcatattcaaactgaca

SEQ ID NO: 141 - DNA, 27 bp, Artificial Sequence
  Description of Artificial Sequence Synthetic primer
ggctacagaggaagaggcaaagatagg

SEQ ID NO: 142 - DNA, 25 bp, Artificial Sequence
  Description of Artificial Sequence Synthetic primer
gcctrggtttrcagagaagcagaca

SEQ ID NO: 143 - DNA, 29 bp, Artificial Sequence
  Description of Artificial Sequence Synthetic primer
agaaatttaggaagtcagaaagctgctca

SEQ ID NO: 144 - DNA, 25 bp, Artificial Sequence
  Description of Artificial Sequence Synthetic primer
gcctaggtttacagagaagcagaca

SEQ ID NO: 145 - DNA, 15 bp, Artificial Sequence
  Description of Artificial Sequence Synthetic oligonucleotide
gcctccctcgcgcca

SEQ ID NO: 146 - DNA, 22 bp, Artificial Sequence
  Description of Artificial Sequence Synthetic oligonucleotide
acactgacgacatggttctaca

SEQ ID NO: 147 - DNA, 4 bp, Artificial Sequence
  Description of Artificial Sequence Synthetic oligonucleotide
tcag

SEQ ID NO: 148 - DNA, 10 bp, Artificial Sequence
  Description of Artificial Sequence Synthetic oligonucleotide
acgagtgcgt